(19) United States
(12) Patent Application Publication
(10) Pub. No.: US 2022/0162599 A1
(43) Pub. Date: May 26, 2022

(54) METHODS AND COMPOSITIONS FOR MODULATING SPLICING OF ALTERNATIVE INTRONS

(71) Applicant: Stoke Therapeutics, Inc., Bedford, MA (US)

(72) Inventor: Isabel Aznarez, Boston, MA (US)

(21) Appl. No.: 17/518,337

(22) Filed: Nov. 3, 2021

Related U.S. Application Data
(63) Continuation of application No. PCT/US2020/029953, filed on Apr. 24, 2020.
(60) Provisional application No. 62/839,572, filed on Apr. 26, 2019.

(51) Int. Cl. C12N 15/11; A61P 25/02; A61P 27/02; A61P 3/00; A61P 35/00; C12N 15/113; A61K 31/7088

(52) U.S. Cl. CPC C12N 15/111; A61P 25/02; A61P 27/02; A61K 31/7088; A61P 35/00; C12N 15/1138; A61P 3/00

(57) ABSTRACT

Provided herein are methods and compositions for modulating expression of a target protein or a target RNA by modulating splicing pre-mRNA and for treating diseases or conditions associated with expression level of the target protein or the target RNA.

### CROSS-REFERENCE

This application is a continuation of International Application No. PCT/US2020/029953, filed Apr. 24, 2020, which claims the benefit of U.S. Provisional Application No. 62/839,572, filed on Apr. 26, 2019, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Nov. 3, 2021, is named 47991_726_301_SL.txt and is 1,023,500 bytes in size.

### BACKGROUND OF THE INVENTION

Alternative splicing events of pre-mRNAs encoded by genes can lead to non-productive mRNA transcripts which in turn can lead to reduced protein expression. Therapeutic agents which can target the alternative splicing events of pre-mRNAs encoded by genes can increase the expression level of functional proteins in patients and/or inhibit aberrant protein expression. Such therapeutic agents can be used to treat a condition that would benefit from increase of protein expression or treat a disease caused by altered expression level of a protein, for example, a condition or a disease caused by protein deficiency. One such example is programmed death-ligand 1 (PD-L1, or CD274), a ligand of programmed cell death-1 (PD-1, CD279).

PD-1 (CD279) is a critical immunoregulatory checkpoint molecule that exerts profound effects on adaptive immune responses and, in particular, on T cell function (Keir et al., 2008, Ann. Rev. Immunol., 26: 677-704). Its ligands, PD-L1 (CD274) and, to a more limited extent, PD-L2, are expressed both on specialized immune cells and in tissues. PD-L1 and PD-L2 interact with PD-1 to send inhibitory signals to invading T cells, inducing immune regulation, anergy, regulatory phenotypes and control of ‘attack.’ Expression of PD-1 and PD-L1 are upregulated in an inflammatory milieu, for example, upon T cell activation in the case of PD-1, and exposure to inflammatory cytokines in the case of PD-L1 and constitute an endogenous ‘brake’ or checkpoint. PD-L1 is also upregulated in a broad group of tumors, where its presence suppresses anti-tumor T cell responses.

PD-1:PD-L1 interaction is a critical mediator of peripheral immune tolerance, the suppression of T cells that inappropriately attack self-tissues, and further, has been shown to modulate established autoimmunity in animal models. Models of inflammatory disease where PD-L1 has been shown to be important include the non-obese diabetic (NOD) model of autoimmune diabetes, the experimental autoimmune encephalomyelitis (EAE) model of multiple sclerosis, the collagen-induced arthritis model of rheumatoid arthritis, multiple models of inflammatory bowel disease (ulcerative colitis, Crohn's disease), models of transplantation and graft versus host disease, autoimmune uveitis and numerous other settings, suggesting a broad role for the PD-1:PD-L1 pathway in inflammatory diseases (See Keir, 2008; Gianchecchi, 2013 for reviews). In humans, genetic polymorphisms in PD-1 have been associated with systemic lupus erythematosus, type 1 diabetes, rheumatoid arthritis, Grave's disease, and multiple sclerosis (Okazaki & Honjo, International Immunology, 2007).

Certain studies suggest that ectopic or overexpression of PD-L1 can regulate and suppress pathogenic immune responses, including studies of NOD mice, EAE, contact hypersensitivity, and lupus nephritis.

### SUMMARY OF THE INVENTION

Described herein is a method of modulating expression of a target protein or a target RNA by cells having an alternative-intron-containing pre-mRNA (AIC pre-mRNA), the AIC pre-mRNA comprising a alternative-intron, a first portion of an exon flanking a 5′ splice site of the alternative-intron, a second portion of the exon flanking a 3′ splice site of the alternative-intron, the method comprising contacting the cells with a therapeutic agent that binds to a targeted region of the AIC pre-mRNA encoding the target protein or the target RNA, whereby splicing of the alternative-intron from the AIC pre-mRNA encoding the target protein or the target RNA is modulated, thereby modulating a level of processed mRNA encoding the target protein or the target RNA, and modulating the expression of the target protein or the target RNA in the cells.

Described herein is a method of treating a disease or a condition in a subject in need thereof by modulating expression of a target protein or a target RNA in a cell of the subject, comprising: contacting a cell of the subject with a therapeutic agent that modulates splicing of an alternative-intron from an alternative-intron-containing pre-mRNA (AIC pre-mRNA) encoding the target protein or the target RNA, wherein the AIC pre-mRNA comprises the alternative-intron, a first portion of an exon flanking a 5′ splice site of the alternative-intron, a second portion of the exon flanking a 3′ splice site of the alternative-intron, wherein the therapeutic agent binds to a targeted region of the AIC pre-mRNA encoding the target protein or the target RNA, whereby splicing of the alternative-intron from the AIC pre-mRNA encoding the target protein or the target RNA is modulated, thereby modulating a level of processed mRNA encoding the target protein or the target RNA, and modulating the expression of the target protein or the target RNA in the cell of the subject.

In some embodiments, modulating the expression of the target protein in the cell comprises increasing the expression of the target protein in the cell. In some embodiments, modulating the level of processed mRNA encoding the target protein comprises increasing the level of processed mRNA encoding the target protein. In some embodiments, inclusion of the alternative-intron from the AIC pre-mRNA encoding the target protein is increased. In some embodiments, modulating the level of processed mRNA encoding the target protein comprises modulating the level of processed mRNA that comprises the alternative-intron, the first portion of the exon and the second portion of the exon.

In some embodiments, the therapeutic agent is a small molecule. In some embodiments, the therapeutic agent is an antisense oligomer (ASO) complementary to the targeted region of the AIC pre-mRNA. In some embodiments, the therapeutic agent is at least 75%, at least 80%, at least 85%, at least 90%, at least 95%, at least 98%, at least 99%, or 100%, complementary to the targeted region of the AIC pre-mRNA encoding the target protein. In some embodiments, at least a portion of the targeted region of the AIC pre-mRNA is within an intron upstream of the first portion of the exon. In some embodiments, at least a portion of the targeted region of the AIC pre-mRNA is within an intron downstream of the second portion of the exon. In some embodiments, at least a portion of the targeted region of the AIC pre-mRNA is within the alternative-intron. In some embodiments, the target protein produced is a fully functional protein. In some embodiments, the target RNA produced is a functional RNA. In some embodiments, the target RNA produced is a fully functional RNA.

In some embodiments, the target protein is PD-L1 (CD274). In some embodiments, the targeted region of the AIC pre-mRNA to which the therapeutic agent binds is located within exon 4 of CD274. In some embodiments, the therapeutic agent binds to a targeted region of a CD274 (PD-L1) AIC pre-mRNA, wherein the targeted region is within a sequence selected from SEQ ID NOs: 68, 69, and 71-76. In some embodiments, the therapeutic agent modulates splicing of an alternative intron from an exon of the AIC pre-mRNA of CD274, wherein the exon comprises a sequence with at least 80%, 85%, 90%, 92%, 95%, 97%, 99% or 100% sequence identity to SEQ ID NO: 68. In some embodiments, the therapeutic agent modulates splicing of an alternative intron from an exon of the AIC pre-mRNA of CD274, wherein the AIC pre-mRNA comprises a sequence with at least 80%, 85%, 90%, 92%, 95%, 97%, 99% or 100% sequence identity to any one of SEQ ID NOs: 71-76.

In some embodiments, the target protein is Rho GTPase-activating protein 23 (ARHGAP23). In some embodiments, the targeted region of the AIC pre-mRNA to which the therapeutic agent binds is located within an exon of ARHGAP23. In some embodiments, the therapeutic agent binds to a targeted region of a ARHGAP23 AIC pre-mRNA, wherein the targeted region is within a sequence selected from SEQ ID NOs: 77 and 91. In some embodiments, the therapeutic agent modulates splicing of an alternative intron from the AIC pre-mRNA of ARHGAP23, wherein the alternative intron comprises a sequence with at least 80%, 85%, 90%, 92%, 95%, 97%, 99% or 100% sequence identity to SEQ ID NO: 77. In some embodiments, the therapeutic agent modulates splicing of an alternative intron from an exon of the AIC pre-mRNA of ARHGAP23, wherein the exon comprises a sequence with at least 80%, 85%, 90%, 92%, 95%, 97%, 99% or 100% sequence identity to SEQ ID NO: 91.

In some embodiments, the target protein is bromodomain containing 1 (BRD1). In some embodiments, the targeted region of the AIC pre-mRNA to which the therapeutic agent binds is located within an exon of BRD1. In some embodiments, the therapeutic agent binds to a targeted region of a BRD1 AIC pre-mRNA, wherein the targeted region is within a sequence selected from SEQ ID NOs: 78 and 92. In some embodiments, the therapeutic agent modulates splicing of an alternative intron from the AIC pre-mRNA of BRD1, wherein the alternative intron comprises a sequence with at least 80%, 85%, 90%, 92%, 95%, 97%, 99% or 100% sequence identity to SEQ ID NO: 78. In some embodiments, the therapeutic agent modulates splicing of an alternative intron from an exon of the AIC pre-mRNA of BRD1, wherein the exon comprises a sequence with at least 80%, 85%, 90%, 92%, 95%, 97%, 99% or 100% sequence identity to SEQ ID NO: 92.

In some embodiments, the target protein is Protocadherin-16 (DCHS1). In some embodiments, the targeted region of the AIC pre-mRNA to which the therapeutic agent binds is located within an exon of DCHS1. In some embodiments, the therapeutic agent binds to a targeted region of a DCHS1 AIC pre-mRNA, wherein the targeted region is within a sequence selected from SEQ ID NOs: 79 and 93. In some embodiments, the therapeutic agent modulates splicing of an alternative intron from the AIC pre-mRNA of DCHS1, wherein the alternative intron comprises a sequence with at least 80%, 85%, 90%, 92%, 95%, 97%, 99% or 100% sequence identity to SEQ ID NO: 79. In some embodiments, the therapeutic agent modulates splicing of an alternative intron from an exon of the AIC pre-mRNA of DCHS1, wherein the exon comprises a sequence with at least 80%, 85%, 90%, 92%, 95%, 97%, 99% or 100% sequence identity to SEQ ID NO: 93.

In some embodiments, the target protein is Erythrocyte Membrane Protein Band 4.1 Like 2 (EPB41L2). In some embodiments, the targeted region of the AIC pre-mRNA to which the therapeutic agent binds is located within an exon of EPB41L2. In some embodiments, the therapeutic agent binds to a targeted region of a EPB41L2 AIC pre-mRNA, wherein the targeted region is within a sequence selected from SEQ ID NOs: 80 and 94. In some embodiments, the therapeutic agent modulates splicing of an alternative intron from the AIC pre-mRNA of EPB41L2, wherein the alternative intron comprises a sequence with at least 80%, 85%, 90%, 92%, 95%, 97%, 99% or 100% sequence identity to SEQ ID NO: 80. In some embodiments, the therapeutic agent modulates splicing of an alternative intron from an exon of the AIC pre-mRNA of EPB41L2, wherein the exon comprises a sequence with at least 80%, 85%, 90%, 92%, 95%, 97%, 99% or 100% sequence identity to SEQ ID NO: 94.

In some embodiments, the target protein is Glutathione peroxidase 8 (GPX8). In some embodiments, the targeted region of the AIC pre-mRNA to which the therapeutic agent binds is located within an exon of GPX8. In some embodiments, the therapeutic agent binds to a targeted region of a GPX8 AIC pre-mRNA, wherein the targeted region is within a sequence selected from SEQ ID NOs: 81 and 95. In some embodiments, the therapeutic agent modulates splicing of an alternative intron from the AIC pre-mRNA of GPX8, wherein the alternative intron comprises a sequence with at least 80%, 85%, 90%, 92%, 95%, 97%, 99% or 100% sequence identity to SEQ ID NO: 81. In some embodiments, the therapeutic agent modulates splicing of an alternative intron from an exon of the AIC pre-mRNA of GPX8, wherein the exon comprises a sequence with at least 80%, 85%, 90%, 92%, 95%, 97%, 99% or 100% sequence identity to SEQ ID NO: 95.

In some embodiments, the target protein is Human immunodeficiency virus type I enhancer-binding protein 3 (HIVEP3). In some embodiments, the targeted region of the AIC pre-mRNA to which the therapeutic agent binds is located within an exon of HIVEP3. In some embodiments, the therapeutic agent binds to a targeted region of a HIVEP3 AIC pre-mRNA, wherein the targeted region is within a sequence selected from SEQ ID NOs: 82 and 96. In some embodiments, the therapeutic agent modulates splicing of an alternative intron from the AIC pre-mRNA of HIVEP3, wherein the alternative intron comprises a sequence with at least 80%, 85%, 90%, 92%, 95%, 97%, 99% or 100% sequence identity to SEQ ID NO: 82. In some embodiments, the therapeutic agent modulates splicing of an alternative intron from an exon of the AIC pre-mRNA of HIVEP3, wherein the exon comprises a sequence with at least 80%, 85%, 90%, 92%, 95%, 97%, 99% or 100% sequence identity to SEQ ID NO: 96.

In some embodiments, the target protein is Inversin (INVS). In some embodiments, the targeted region of the AIC pre-mRNA to which the therapeutic agent binds is located within an exon of INVS. In some embodiments, the therapeutic agent binds to a targeted region of an INVS AIC pre-mRNA, wherein the targeted region is within a sequence selected from SEQ ID NOs: 83 and 97. In some embodiments, the therapeutic agent modulates splicing of an alternative intron from the AIC pre-mRNA of INVS, wherein the alternative intron comprises a sequence with at least 80%, 85%, 90%, 92%, 95%, 97%, 99% or 100% sequence identity to SEQ ID NO: 83. In some embodiments, the therapeutic agent modulates splicing of an alternative intron from an exon of the AIC pre-mRNA of INVS, wherein the exon comprises a sequence with at least 80%, 85%, 90%, 92%, 95%, 97%, 99% or 100% sequence identity to SEQ ID NO: 97.

In some embodiments, the target protein is Dyslexia-associated protein KIAA0319 (KIAA0319). In some embodiments, the targeted region of the AIC pre-mRNA to which the therapeutic agent binds is located within an exon of KIAA0319. In some embodiments, the therapeutic agent binds to a targeted region of a KIAA0319 AIC pre-mRNA, wherein the targeted region is within a sequence selected from SEQ ID NOs: 84 and 98. In some embodiments, the therapeutic agent modulates splicing of an alternative intron from the AIC pre-mRNA of KIAA0319, wherein the alternative intron comprises a sequence with at least 80%, 85%, 90%, 92%, 95%, 97%, 99% or 100% sequence identity to SEQ ID NO: 84. In some embodiments, the therapeutic agent modulates splicing of an alternative intron from an exon of the AIC pre-mRNA of KIAA0319, wherein the exon comprises a sequence with at least 80%, 85%, 90%, 92%, 95%, 97%, 99% or 100% sequence identity to SEQ ID NO: 98.

In some embodiments, the target protein is NLR Family Apoptosis Inhibitory Protein (NAIP). In some embodiments, the targeted region of the AIC pre-mRNA to which the therapeutic agent binds is located within an exon of NAIP. In some embodiments, the therapeutic agent binds to a targeted region of a NAIP AIC pre-mRNA, wherein the targeted region is within a sequence selected from SEQ ID NOs: 85 and 99. In some embodiments, the therapeutic agent modulates splicing of an alternative intron from the AIC pre-mRNA of NAIP, wherein the alternative intron comprises a sequence with at least 80%, 85%, 90%, 92%, 95%, 97%, 99% or 100% sequence identity to SEQ ID NO: 85. In some embodiments, the therapeutic agent modulates splicing of an alternative intron from an exon of the AIC pre-mRNA of NAIP, wherein the exon comprises a sequence with at least 80%, 85%, 90%, 92%, 95%, 97%, 99% or 100% sequence identity to SEQ ID NO: 99.

In some embodiments, the target protein is Patched 2 (PTCH2). In some embodiments, the targeted region of the AIC pre-mRNA to which the therapeutic agent binds is located within an exon of PTCH2. In some embodiments, the therapeutic agent binds to a targeted region of a PTCH2 AIC pre-mRNA, wherein the targeted region is within a sequence selected from SEQ ID NOs: 86 and 100. In some embodiments, the therapeutic agent modulates splicing of an alternative intron from the AIC pre-mRNA of PTCH2, wherein the alternative intron comprises a sequence with at least 80%, 85%, 90%, 92%, 95%, 97%, 99% or 100% ho sequence identity to SEQ ID NO: 86. In some embodiments, the therapeutic agent modulates splicing of an alternative intron from an exon of the AIC pre-mRNA of PTCH2, wherein the exon comprises a sequence with at least 80%, 85%, 90%, 92%, 95%, 97%, 99% or 100% sequence identity to SEQ ID NO: 100.

In some embodiments, the target protein is Protein Tyrosine Phosphatase Receptor Type Z1 (PTPRZ1). In some embodiments, the targeted region of the AIC pre-mRNA to which the therapeutic agent binds is located within an exon of PTPRZ1. In some embodiments, the therapeutic agent binds to a targeted region of a PTPRZ1 AIC pre-mRNA, wherein the targeted region is within a sequence selected from SEQ ID NOs: 87 and 101. In some embodiments, the therapeutic agent modulates splicing of an alternative intron from the AIC pre-mRNA of PTPRZ1, wherein the alternative intron comprises a sequence with at least 80%, 85%, 90%, 92%, 95%, 97%, 99% or 100% sequence identity to SEQ ID NO: 87. In some embodiments, the therapeutic agent modulates splicing of an alternative intron from an exon of the AIC pre-mRNA of PTPRZ1, wherein the exon comprises a sequence with at least 80%, 85%, 90%, 92%, 95%, 97%, 99% or 100% sequence identity to SEQ ID NO: 101.

In some embodiments, the target protein is SON. In some embodiments, the targeted region of the AIC pre-mRNA to which the therapeutic agent binds is located within an exon of SON. In some embodiments, the therapeutic agent binds to a targeted region of a SON AIC pre-mRNA, wherein the targeted region is within a sequence selected from SEQ ID NOs: 88, 89, 102 and 103. In some embodiments, the therapeutic agent modulates splicing of an alternative intron from the AIC pre-mRNA of SON, wherein the alternative intron comprises a sequence with at least 80%, 85%, 90%, 92%, 95%, 97%, 99% or 100% sequence identity to SEQ ID NO: 88 or SEQ ID NO: 89. In some embodiments, the therapeutic agent modulates splicing of an alternative intron from an exon of the AIC pre-mRNA of SON, wherein the exon comprises a sequence with at least 80%, 85%, 90%, 92%, 95%, 97%, 99% or 100% sequence identity to SEQ ID NO: 102 or SEQ ID NO: 103.

In some embodiments, the target protein is Zinc finger CCHC domain-containing protein 2 (ZCCHC2). In some embodiments, the targeted region of the AIC pre-mRNA to which the therapeutic agent binds is located within an exon of ZCCHC2. In some embodiments, the therapeutic agent binds to a targeted region of a ZCCHC2 AIC pre-mRNA, wherein the targeted region is within a sequence selected from SEQ ID NOs: 90 and 104. In some embodiments, the therapeutic agent modulates splicing of an alternative intron from the AIC pre-mRNA of ZCCHC2, wherein the alternative intron comprises a sequence with at least 80%, 85%, 90%, 92%, 95%, 97%, 99% or 100% sequence identity to SEQ ID NO: 90. In some embodiments, the therapeutic agent modulates splicing of an alternative intron from an exon of the AIC pre-mRNA of ZCCHC2, wherein the exon comprises a sequence with at least 80%, 85%, 90%, 92%, 95%, 97%, 99% or 100% sequence identity to SEQ ID NO: 104.

In some embodiments, the disease or the condition is an immune disease or an immune disorder. In some embodiments, the immune disease or the immune disorder is an autoimmune disease or an autoimmune disorder, an inflammatory disease or an inflammatory disorder, a chronic infection, graft-versus-host disease (GVHD), a transplant rejection, or a T cell proliferative disorder. In some embodiments, the immune disease or the immune disorder is an autoimmune disease or an autoimmune disorder or an inflammatory disease or an inflammatory disorder selected from: multiple sclerosis, inflammatory bowel disease, autoimmune hepatitis, kidney inflammation, rheumatoid arthritis, psoriasis, lupus nephritis, corneal transplant, and uveitis.

In some embodiments, the disease or the condition is caused by a deficient amount or activity of the target protein. In some embodiment, the disease or the condition is treated or prevented by an increase in the amount or activity of the target protein. In some embodiments, the disease or the condition is induced by a loss-of-function mutation in the target protein.

In some embodiments, the therapeutic agent increases the level of processed mRNA encoding the target protein in the cell. In some embodiments, the level of processed mRNA encoding the target protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell.

In some embodiments, the therapeutic agent increases the expression of the target protein in the cell. In some embodiments, the level of the target protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of the target protein in a control cell.

In some embodiments, inclusion of the alternative-intron from the AIC pre-mRNA encoding the target protein is decreased. In some embodiments, the therapeutic agent decreases the level of processed mRNA encoding the target protein in the cell. In some embodiments, the level of processed mRNA encoding the target protein in the cell contacted with the therapeutic agent is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell.

In some embodiments, the therapeutic agent decreases the expression of the target protein in the cell. In some embodiments, the level of the target protein in the cell contacted with the therapeutic agent is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of the target protein in a control cell.

In some embodiments, the therapeutic agent comprises a backbone modification comprising a phosphorothioate linkage or a phosphorodiamidate linkage. In some embodiments, the therapeutic agent comprises a phosphorodiamidate morpholino, a locked nucleic acid, a peptide nucleic acid, a 2′-O-methyl, a 2′-Fluoro, or a 2′-O-methoxyethyl moiety. In some embodiments, the therapeutic agent comprises at least one modified sugar moiety. In some embodiments, each sugar moiety is a modified sugar moiety. In some embodiments, the therapeutic agent consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

In some embodiments, the method further comprises assessing mRNA level or expression level of the target protein. In some embodiments, the subject is a human. In some embodiments, the subject is a non-human animal. In some embodiments, the subject is a fetus, an embryo, or a child. In some embodiments, the cell or the cells is ex vivo, or in a tissue, or an organ ex vivo.

In some embodiments, the therapeutic agent is administered to the subject by intracerebroventricular injection, intraperitoneal injection, intramuscular injection, intrathecal injection, subcutaneous injection, oral administration, synovial injection, intravitreal administration, subretinal injection, topical application, implantation, or intravenous injection.

In some embodiments, the splicing of the alternative-intron from the AIC pre-mRNA encoding the target protein or the target RNA produces a processed mRNA with a premature termination codon (PTC). In some embodiments, the splicing of the alternative-intron from the AIC pre-mRNA encoding the target protein or the target RNA produces a processed mRNA with a premature termination codon (PTC) that does not encode a functional target protein or does not encode a functional RNA. In some embodiments, the splicing of the alternative-intron from the AIC pre-mRNA encoding the target protein or the target RNA produces a processed mRNA with a premature termination codon (PTC) that encodes a non-functional target protein or a non-functional target RNA. In some embodiments, the splicing of the alternative-intron from the AIC pre-mRNA encoding the target protein or the target RNA produces a processed mRNA that has a lower translation or expression efficiency for producing the target protein as compared to a corresponding processed mRNA that is otherwise identical but comprises the alternative-intron. In some embodiments, the splicing of the alternative-intron from the AIC pre-mRNA encoding the target protein or the target RNA produces a processed mRNA that undergoes non-sense mediated decay (NMD). In some embodiments, the splicing of the alternative-intron from the AIC pre-mRNA encoding the target protein or the target RNA produces a processed mRNA that undergoes non-sense mediated decay (NMD).

Provided herein is a therapeutic agent for use in methods described herein. Provided herein is a pharmaceutical composition comprising the therapeutic agent described herein and a pharmaceutically acceptable excipient.

Provided herein is a method of treating a subject in need thereof, comprising administering the pharmaceutical composition of claim 48 by intracerebroventricular injection, intraperitoneal injection, intramuscular injection, intrathecal injection, subcutaneous injection, oral administration, synovial injection, intravitreal administration, subretinal injection, topical application, implantation, or intravenous injection to the subject.

Provided herein is a composition comprising a therapeutic agent for use in a method of modulating expression of a target protein or a target RNA by cells to treat a disease or a condition in a subject in need thereof, associated with an aberrant protein or an aberrant RNA in the subject, wherein the aberrant protein or aberrant RNA is aberrant in amount or activity in the subject, wherein the therapeutic agent modulates splicing of an alternative-intron-containing pre-mRNA (AIC pre-mRNA) encoding the target protein or the target RNA, wherein the target protein is: (a) the aberrant protein; (b) a protein which functionally activates or deactivates cellular signaling mechanisms to alter cellular activity associated with the disease or condition (c) a protein which functionally augments or replaces the aberrant protein in the subject; or (d) a protein which functionally decreases or inhibits the aberrant protein in the subject; and wherein the target RNA is: (a) the aberrant RNA; (b) an RNA which functionally activates or deactivates cellular signaling mechanisms to alter cellular activity associated with the disease or condition; (c) an RNA which functionally augments or replaces the aberrant RNA in the subject; or (d) an RNA which functionally decreases or inhibits the aberrant RNA in the subject; wherein the AIC pre-mRNA comprising an alternative-intron, a first portion of an exon flanking a 5′ splice site of the alternative-intron, a second portion of the exon flanking a 3′ splice site of the alternative-intron, and whereby splicing of the alternative-intron from the AIC pre-mRNA encoding the target protein or the target RNA is modulated, thereby modulating production or activity of the target protein or the target RNA in the subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings.

FIG. 1 shows a schematic of the generation of different splice variants

FIGS. 2A-C show measurements of different mRNA isoforms in different cells and conditions

FIG. 3 shows a schematic of an ASO walk.

FIGS. 4A-C show measurements of mRNA isoforms and protein expression in cells transfected with different ASOs.

FIGS. 5A-D show measurements of ASO efficacy.

FIGS. 6A-C show measurements of ASO efficacy.

### SEQUENCES

This application includes nucleotide sequences SEQ ID NO: 1-3651, listed in Tables 1-3. The nucleotide sequences set forth as SEQ ID NOS 1-67 in Table 1 are examples of antisense oligomers (ASOs) useful in the methods described herein. The nucleotide sequences set forth as SEQ ID NOs: 68 in Table 2 are examples of sequences that can be targeted by ASOs by the methods described herein. The nucleotide sequence set forth in Table 2 as SEQ ID NO: 69 is a CD274 mRNA sequence. The nucleotide sequence set forth in Table 2 as SEQ ID NO: 70 is a CD274 amino acid sequence. The nucleotide sequence set forth in Table 2 as SEQ ID NO: 71 is CD274 genomic sequence. Table 2 as SEQ ID NOs: 72-76 are pre-mRNA sequences. The nucleotide sequences set forth as SEQ ID NOs: 77-90 in Table 2 are alternative introns to be targeted in genes listed in the table. The nucleotide sequences set forth as SEQ ID NOs: 91-104 in Table 2 are exons to be targeted in genes listed in the table. The nucleotide sequences set forth as SEQ ID NOS 105-3651 in Table 2 are examples of antisense oligomers (ASOs) useful in the methods described herein. Upper case letters represent exon sequence and lower-case letters represent intron sequence.

### DETAILED DESCRIPTION OF THE INVENTION

### Splicing and Alternative Splice Sites

Intervening sequences or introns are removed by a large and highly dynamic RNA-protein complex termed the spliceosome, which orchestrates complex interactions between primary transcripts, small nuclear RNAs (snRNAs), and a large number of proteins. Spliceosomes assemble ad hoc on each intron in an ordered manner, starting with recognition of the 5′ splice site (5′ss) by U1 snRNA or the 3′splice site (3′ss) by the U2 pathway, which involves binding of the U2 auxiliary factor (U2AF) to the 3′ss region to facilitate U2 binding to the branch point sequence (BPS). U2AF is a stable heterodimer composed of a U2AF2-encoded 65-kD subunit (U2AF65), which binds the polypyrimidine tract (PPT), and a U2AF1-encoded 35-kD subunit (U2AF35), which interacts with highly conserved AG dinucleotides at 3′ss and stabilizes U2AF65 binding. In addition to the BPS/PPT unit and 3′ss/5′ss, accurate splicing requires auxiliary sequences or structures that activate or repress splice site recognition, known as intronic or exonic splicing enhancers or silencers. These elements allow genuine splice sites to be recognized among a vast excess of cryptic or pseudo-sites in the genome of higher eukaryotes, which have the same sequences but outnumber authentic sites by an order of magnitude.

The decision of whether to splice or not to splice can be typically modeled as a stochastic rather than deterministic process, such that even the most defined splicing signals can sometimes splice incorrectly. However, under normal conditions, pre-mRNA splicing proceeds at surprisingly high fidelity. This is attributed in part to the activity of adjacent cis-acting auxiliary exonic and intronic splicing regulatory elements (ESRs or ISRs). Typically, these functional elements are classified as either exonic or intronic splicing enhancers (ESEs or ISEs) or silencers (ESSs or ISSs) based on their ability to stimulate or inhibit splicing, respectively. Although there is now evidence that some auxiliary cis-acting elements may act by influencing the kinetics of spliceosome assembly, such as the arrangement of the complex between U1 snRNP and the 5′ss, it seems very likely that many elements function in concert with trans-acting RNA-binding proteins (RBPs). For example, the serine- and arginine-rich family of RBPs (SR proteins) is a conserved family of proteins that have a key role in defining exons. SR proteins promote exon recognition by recruiting components of the pre-spliceosome to adjacent splice sites or by antagonizing the effects of ESSs in the vicinity. The repressive effects of ESSs can be mediated by members of the heterogeneous nuclear ribonucleoprotein (hnRNP) family and can alter recruitment of core splicing factors to adjacent splice sites. In addition to their roles in splicing regulation, silencer elements are suggested to have a role in repression of pseudo-exons, sets of decoy intronic splice sites with the typical spacing of an exon but without a functional open reading frame. ESEs and ESSs, in cooperation with their cognate trans-acting RBPs, represent important components in a set of splicing controls that specify how, where, and when mRNAs are assembled from their precursors.

The sequences marking the exon-intron boundaries are degenerate signals of varying strengths that can occur at high frequency within human genes. In multi-exon genes, different pairs of splice sites can be linked together in many different combinations, creating a diverse array of transcripts from a single gene. This is commonly referred to as alternative pre-mRNA splicing. Although most mRNA isoforms produced by alternative splicing can be exported from the nucleus and translated into functional polypeptides, different mRNA isoforms from a single gene can vary greatly in their translation efficiency. Those mRNA isoforms with premature termination codons (PTCs) at least 50 bp upstream of an exon junction complex are likely to be targeted for degradation by the nonsense-mediated mRNA decay (NMD) pathway. Mutations in traditional (BPS/PPT/3′ss/5′ss) and auxiliary splicing motifs can cause aberrant splicing, such as exon skipping or cryptic (or pseudo-) exon inclusion or splice-site activation and contribute significantly to human morbidity and mortality. Both aberrant and alternative splicing patterns can be influenced by natural DNA variants in exons and introns.

Cryptic (or pseudo-) splice sites have the same splicing recognition sequences as genuine splice sites but are not used in splicing reactions. They outnumber genuine splice sites in the human genome by an order of a magnitude and are normally repressed by thus far poorly understood molecular mechanisms. Cryptic 5′ splice sites have the consensus NNN/GUNNNN or NNN/GCNNNN where N is any nucleotide and is the exon-intron boundary. Cryptic 3′ splice sites have the consensus NAG/N. Their activation is positively influenced by surrounding nucleotides that make them more similar to the optimal consensus of authentic splice sites, namely MAG/GURAGU and YAG/G, respectively, where M is C or A, R is G or A, and Y is C or U. Activation of cryptic (or pseudo-) splice sites can be influenced or determined by the balance between the intrinsic strength of aberrant splice sites and their authentic counterparts, the availability of traditional signals in the vicinity of mutated splice sites, exon and intron size, the nature of mutation and by disrupting or creating ESEs, ESSs, ISEs, or ISSs.

Splice sites and their regulatory sequences can be readily identified by a skilled person using suitable algorithms publicly available, listed, for example, in Kralovicova, J. and Vorechovsky, I. (2007) Global control of aberrant splice site activation by auxiliary splicing sequences: evidence for a gradient in exon and intron definition. Nucleic Acids Res., 35, 6399-6413.

An exon sequence may contain both 5′ and 3′ alternative splice sites and this may promote RNA-binding proteins, such as U2AF, to the alternative splice sites within the exon sequence, resulting in splicing of a portion of the exon from the pre-mRNA and producing a processed mRNA missing the portion of the exon. The region or sequence between a 5′ alternative splice site and a 3′ alternative splice site within an exon can be referred to an alternative-intron. A pre-mRNA with 5′ and 3′ alternative splice sites located within an exon sequence can be referred to an alternative-intron-containing pre-mRNA (AIC pre-mRNA). In some embodiments, an agent may bind to an alternative splice site or splicing regulatory sequence to prevent binding of RNA-binding proteins and thereby inhibit splicing of an alternative-intron. In some embodiments, an agent may bind to an alternative splice site or splicing regulatory sequence to promote binding of RNA-binding proteins and thereby enhance splicing of an alternative-intron.

Provided herein are methods and compositions that can modulate aberrant splicing events at alternative splice sites to modulate the level of functional mRNAs encoded by a target gene and expression level of the protein encoded by the target gene. The methods and compositions of the invention can be used to modulate the expression level of a target protein in a subject or in a cell of a subject. For example, the methods and compositions of the invention can be used to increase or decrease the expression level of a target protein (without changing the protein sequence) in a subject or in a cell of a subject. The methods and compositions of the invention can be used to modulate the level of processed mRNA encoding a target protein in a subject or a cell of a subject. For example, the methods and compositions of the invention can be used to increase or decrease the level of processed mRNA encoding a target protein in a subject or a cell of a subject.

Provided herein is a method of modulating expression of a target protein or a target RNA by cells having an alternative-intron-containing pre-mRNA (AIC pre-mRNA). In one aspect, the AIC pre-mRNA comprises an alternative-intron, a first portion of an exon flanking a 5′ splice site of the alternative-intron, and a second portion of the exon flanking a 3′ splice site of the alternative-intron. In one aspect, the method comprises contacting the cells with a therapeutic agent that binds to a targeted region of the AIC pre-mRNA encoding the target protein or the target RNA, whereby splicing of the alternative-intron from the AIC pre-mRNA encoding the target protein or the target RNA is modulated, thereby modulating a level of processed mRNA encoding the target protein or the target RNA, and modulating the expression of the target protein or the target RNA in the cells.

Provided herein is a method of treating a disease or a condition in a subject in need thereof by modulating expression of a target protein or a target RNA in a cell of the subject. In one aspect, the method comprises contacting a cell of the subject with a therapeutic agent that modulates splicing of an alternative-intron from an alternative-intron-containing pre-mRNA (AIC pre-mRNA) encoding the target protein or the target RNA, wherein the AIC pre-mRNA comprises the alternative-intron, a first portion of an exon flanking a 5′ splice site of the alternative-intron, a second portion of the exon flanking a 3′ splice site of the alternative-intron, wherein the therapeutic agent binds to a targeted region of the AIC pre-mRNA encoding the target protein or the target RNA, whereby splicing of the alternative-intron from the AIC pre-mRNA encoding the target protein or the target RNA is modulated, thereby modulating a level of processed mRNA encoding the target protein or the target RNA, and modulating the expression of the target protein or the target RNA in the cell of the subject.

In some embodiments, modulating the expression of the target protein in the cell comprises increasing the expression of the target protein in the cell. In some embodiments, the expression of the target protein in the cell is increased by at least 10, 20, 30, 40, 50, 60, 80, 100, 150, 200, 250, 300, 350, 400, 450, 500, or 1000%, compared to the expression of the target protein that is not modulated. In some embodiments, the expression of the target protein in the cell is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the expression of the target protein that is not modulated.

In some embodiments, modulating the level of processed mRNA encoding the target protein comprises increasing the level of processed mRNA encoding the target protein. In some embodiments, the level of processed mRNA encoding the target protein in the cell is increased by at least 10, 20, 30, 40, 50, 60, 80, 100, 150, 200, 250, 300, 350, 400, 450, 500, or 1000%, compared to the level of processed mRNA encoding the target protein that is not modulated. In some embodiments, the level of processed mRNA encoding the target protein in the cell is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein that is not modulated.

In some embodiments, splicing of the alternative-intron from the AIC pre-mRNA encoding the target protein is inhibited. In some embodiments, modulating the level of processed mRNA encoding the target protein comprises modulating the level of processed mRNA that comprises the alternative-intron, the first portion of the exon and the second portion of the exon. In some embodiments, increasing the level of processed mRNA encoding the target protein comprises increasing the level of processed mRNA that comprises the alternative-intron, the first portion of the exon and the second portion of the exon. In some embodiments, decreasing the level of processed mRNA encoding the target protein comprises decreasing the level of processed mRNA that comprises the alternative-intron, the first portion of the exon and the second portion of the exon.

In some embodiments, splicing of the alternative-intron from the AIC pre-mRNA encoding the target protein is promoted. In some embodiments, modulating the expression of the target protein in the cell comprises decreasing the expression of the target protein in the cell. In some embodiments, the expression of the target protein in the cell is decreased by at least 1, 5, 10, 15, 20, 25, 30, 35, 40, 45, 50, 55, 60, 65, 70, 75, 80, 85, 90, 95, 100%, compared to the expression of the target protein that is not modulated. In some embodiments, the expression of the target protein in the cell is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the expression of the target protein that is not modulated.

In some embodiments, modulating the level of processed mRNA encoding the target protein comprises decreasing the level of processed mRNA encoding the target protein. In some embodiments, the level of processed mRNA encoding the target protein in the cell is decreased by at least 1, 5, 10, 15, 20, 25, 30, 35, 40, 45, 50, 55, 60, 65, 70, 75, 80, 85, 90, 95, 100%, compared to the level of processed mRNA encoding the target protein that is not modulated. In some embodiments, the level of processed mRNA encoding the target protein in the cell is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein that is not modulated.

In some embodiments, the therapeutic agent is a small molecule. In some embodiments, the therapeutic agent is an antisense oligomer (ASO) complementary to the targeted region of the AIC pre-mRNA. In some embodiments, the therapeutic agent is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the targeted region of the AIC pre-mRNA encoding the target protein.

In some embodiments, at least a portion of the targeted region of the AIC pre-mRNA is within an intron upstream of the first portion of the exon. In some embodiments, at least a portion of the targeted region of the AIC pre-mRNA is within an intron downstream of the second portion of the exon. In some embodiments, at least a portion of the targeted region of the AIC pre-mRNA is within the alternative-intron. In some embodiments, at least a portion of the targeted region of the AIC pre-mRNA is within the first portion of the exon. In some embodiments, at least a portion of the targeted region of the AIC pre-mRNA is within the second portion of the exon. In some embodiments, at least a portion of the targeted region of the AIC pre-mRNA overlaps with a junction of the intron upstream of the first portion of the exon and the first portion of the exon. In some embodiments, at least a portion of the targeted region of the AIC pre-mRNA overlaps with a junction of the first portion of the exon and the alternative-intron. In some embodiments, at least a portion of the targeted region of the AIC pre-mRNA overlaps with a junction of the alternative-intron and the second portion of the exon. In some embodiments, at least a portion of the targeted region of the AIC pre-mRNA overlaps with a junction of the second portion of the exon and the intron downstream of the second portion of the exon.

In some embodiments, the method is a method of increasing the expression of the target protein by cells of a subject having a AIC pre-mRNA encoding the target protein, wherein the subject has a disease or a disorder caused by a deficient amount or activity of the target protein. In some embodiments, the deficient amount of the target protein is caused by haploinsufficiency of the target protein. In some embodiments, the disease or the disorder is associated with haploinsufficiency of a gene encoding the target protein. In some embodiments, the subject has a first allele encoding a functional target protein, and a second allele from which the target protein is not produced. In some embodiments, the subject has a first allele encoding a functional target protein, and a second allele from which the target protein is produced at a reduced level. In another embodiment, the subject has a first allele encoding a functional target protein, and a second allele encoding a nonfunctional target protein. In another embodiment, the subject has a first allele encoding a functional target protein, and a second allele encoding a partially functional target protein. In any of these embodiments, the ASO binds to a targeted region of the AIC pre-mRNA transcribed from the first allele (encoding functional target protein), thereby inhibiting splicing of the alternative-intron from the AIC pre-mRNA, and causing an increase in the level of mature mRNA encoding functional target protein, and an increase in the expression of the target protein in the cells of the subject.

In some embodiments, the AIC pre-mRNA transcript that encodes the protein that is causative of the disease or condition is targeted by the ASOs described herein. In some embodiments, a AIC pre-mRNA transcript that encodes a protein that is not causative of the disease is targeted by the ASOs. For example, a disease that is a result of a mutation or deficiency of a first protein in a particular pathway may be ameliorated by targeting a AIC pre-mRNA that encodes a second protein, thereby increasing production of the second protein. In some embodiments, the function of the second protein is able to compensate for the mutation or deficiency of the first protein (which is causative of the disease or condition).

In some embodiments, the AIC pre-mRNA transcript encodes a protein that helps alleviate the disease or condition. For example, the AIC pre-mRNA transcript may encode a signaling protein that may activate or deactivate a signaling pathway associated with the disease. The pathway may be related to immune responses and may cause upregulation of immune response, for example, to combat aberrant cell growth or foreign pathogens. The pathway may be related to immune responses and may cause down regulation of immune response to for example, decrease inflammation or aberrant immune responses (i.e. auto-immune or allergic reactions).

In some embodiments, the subject has:

a. a first mutant allele from which
i) the target protein is produced at a reduced level compared to production from a wild-type allele,
ii) the target protein is produced in a form having reduced function compared to an equivalent wild-type protein, or
iii) the target protein or functional RNA is not produced; and

b. a second mutant allele from which
i) the target protein is produced at a reduced level compared to production from a wild-type allele,
ii) the target protein is produced in a form having reduced function compared to an equivalent wild-type protein, and
   wherein the AIC pre-mRNA is transcribed from the first allele and the second allele. In these embodiments, the ASO binds to a targeted region of the AIC pre-mRNA transcribed from the first allele and the second allele, thereby inhibiting splicing of the alternative-intron from the AIC pre-mRNA, and causing an increase in the level of mRNA encoding the target protein and an increase in the expression of the target protein or functional RNA in the cells of the subject. In these embodiments, the target protein or functional RNA having an increase in expression level resulting from inhibiting splicing of the alternative-intron from the AIC pre-mRNA is either in a form having reduced function compared to the equivalent wild-type protein (partially-functional), or having full function compared to the equivalent wild-type protein (fully-functional).

Disclosed herein is a composition comprising a therapeutic agent for use in a method of modulating expression of a target protein or a target RNA by cells to treat a disease or a condition in a subject in need thereof, associated with an aberrant protein or an aberrant RNA in the subject, wherein the aberrant protein or aberrant RNA is aberrant in amount or activity in the subject, wherein the therapeutic agent modulates splicing of an alternative-intron-containing pre-mRNA (AIC pre-mRNA) encoding the target protein or the target RNA,

wherein the target protein is:
(a) the aberrant protein;
(b) a protein which functionally activates or deactivates cellular signaling mechanisms to alter cellular activity associated with the disease or condition;
(c) a protein which functionally augments or replaces the aberrant protein in the subject; or
(d) a protein which functionally decreases or inhibits the aberrant protein in the subject;
   and wherein the target RNA is:
(a) the aberrant RNA;
(b) an RNA which functionally activates or deactivates cellular signaling mechanisms to alter cellular activity associated with the disease or condition;
(c) an RNA which functionally augments or replaces the aberrant RNA in the subject; or
(d) an RNA which functionally decreases or inhibits the aberrant RNA in the subject;
   wherein the AIC pre-mRNA comprising an alternative-intron, a first portion of an exon flanking a 5′ splice site of the alternative-intron, a second portion of the exon flanking a 3′ splice site of the alternative-intron, and whereby splicing of the alternative-intron from the AIC pre-mRNA encoding the target protein or the target RNA is modulated, thereby modulating production or activity of the target protein or the target RNA in the subject.

In some embodiments, the target protein produced is a fully functional protein. In some embodiments, the target RNA produced is a functional RNA. In some embodiments, the target RNA produced is a fully functional RNA. In some embodiments, the target protein is PD-L1 (CD274). In some embodiments, the target RNA is a functional RNA transcribed from CD274 gene and encoding PD-L1 protein. In some embodiments, the target RNA is a fully functional RNA transcribed from CD274 gene and encoding PD-L1 protein.

### PD-L1 Protein

The balance between T cell activation, tolerance, and immunopathology plays an important role in autoimmune diseases such as systemic lupus erythematosus (SLE), type I diabetes, rheumatoid arthritis, Graves disease, and multiple sclerosis (MS), as well as in transplant rejection and graft-versus-host disease (GVHD). Among key regulators of T cell responses are programmed death 1 (PD-1; also known as CD279) and its ligands PD-L1 (CD274) and PD-L2. Interaction between PD-1 and its ligands regulates T cell activation by delivering inhibitory signals, thus regulating the immune response by limiting effector T cell responses and protecting tissues from immune-mediated damage.

First identified as a negative regulator of T cell activation in a mouse model of autoimmunity, the PD-1 protein is a 288 amino acid (aa) type I transmembrane protein that contains one immunoglobulin (Ig) superfamily domain, an immunoreceptor tyrosine-based inhibitory motif (ITIM), and an immunoreceptor tyrosine-based switch motif (ITSM). The PD-1 ligand PD-L1 is a 290 amino acid type I transmembrane protein encoded by the CD274 gene on human chromosome 9 that comprises seven exons. PD-L1 protein has an IgV-like domain, an IgC-like domain, and a short tail of about 30 amino acids of unknown function. Interaction of PD-L1 with the PD-1 receptor is mediated by the IgV-like domain. The human genomic sequence of the CD274 gene is set forth at NCBI Gene ID 29126. The CD274 canonical mRNA sequence is set forth at NCBI Reference Sequence: NM_014143.3.

PD-L1 is constitutively expressed on both hematopoietic and non-hematopoietic cells, including B cells, T cells, plasmacytoid dendritic cells (pDC), mesenchymal stem cells, and vascular endothelium. Expression of PD-L1 is upregulated by both type I and type II interferons (IFNs) and is decreased in the absence of MyD88, TRAF6, and MEK. Binding of PD-L1 to the PD-1 receptor results in phosphorylation of PD-1 cytoplasmic tyrosines, recruitment of SHP-2, and inhibition of PI3K and Akt activity, ultimately leading to inhibition of T-cell receptor (TCR) signaling that can be overcome by CD28 co-stimulation. In the absence of CD28 co-stimulation, PD-1 ligation decreases induction of cytokines such as IFN-γ and cell survival proteins such as Bcl-xL (Keir et al., 2008, and Trabattoni, et al., 2009, J. Immunol. 183: 4984-4993, incorporated by reference herein). In addition to PD-1, B7-1 has been identified as a binding partner of PD-L1. Interaction between B7-1 and PD-L1 occurs through the IgV-like domains and results in induction of an inhibitory signal in T cells, thereby limiting T cell responses.

PD1 and PD-L1 play a role in preventing initiation and progression to autoimmunity and in T cell tolerance in the periphery (Keir et al., 2008, and Trabattoni, et al., 2009). For example, a role of PD-1: PD-L1 interaction in autoimmunity has been demonstrated in mice deficient in PD-1. PD-1:PD-L1 interaction plays a role in both positive and negative T cell selection in the thymus, consistent with a role in central tolerance induction. Negative T cell regulation by PD-1: PD-L1 also plays a role in peripheral tolerance by inhibiting responses of self-reactive T cells and mediating responses of T regulatory cells. As an example, loss of PD-1 or PD-L1 was reported to result in rapid or exacerbated diabetes in a mouse model of autoimmune T-cell mediated diabetes. Administration of anti-PD-1 or anti-PD-L1 mAbs during induction of experimental autoimmune encephalomyelitis (EAE) in a mouse model resulted in accelerated disease onset and severity (summarized by Keir et al., 2008). Studies of the NOD mouse (Li et al, 2015, Diabetes 64:529-540; Wang et al, 2008, Diabetes 57:1861-69), EAE (Hirata et al, 2005, J. Immunology 174:1888-1897), contact hypersensitivity (Ritprajak et al, 2010, J. Immunology 184:4918-4925), and lupus nephritis (Ding et al., 2006, Clinical Immunology 118:258-67) indicate that ectopic expression or overexpression of PD-L1 can regulate and suppress pathogenic immune responses (each incorporated by reference herein). In addition to these ectopic, tissue-based overexpression studies, many further reports have evaluated the effects of soluble PD-L1-Fc or -Ig fusions, which provide additional evidence for the immunoregulatory consequences of exogenous/ectopic PD-L1 administration.

In addition to a role in autoimmunity, interaction of PD-L1 with PD-1 controls organ engraftment and graft-versus-host disease (GVHD). Both PD-1 and PD-L1 are upregulated in alloreactive T cells in transplant recipients and may control alloreactive immune responses. PD-1 is upregulated after the onset of GVHD, and PD-L1 is expressed on most cells in GVHD target organs. Significantly, administration of PD-L1 blocking antibodies accelerated transplant rejection in heart, corneal, and skin transplant models. A central role of PD-L1 in graft tolerance has been demonstrated in a transplant model receiving CTLA-4-Ig treatment to induce tolerance and using CD274−/−mice as transplant donors or recipients. PD-L1 expression in the graft protected from local pathology, and PD-L1 expression in the recipient immune system was required for induction and maintenance of transplantation tolerance. A potential mechanism by which PD-L1 may reduce graft rejection is through induction of T cell apoptosis. Notably, use of PD-1 and PD-L1 blocking antibodies in transplantation models suggested a PD-1 independent role for PD-L1 in promoting tolerance trough induction of alloreactive T cell apoptosis (Keir, et al., 2008). In some embodiments, the methods and compositions of the present invention increase levels of PD-L1 thereby resulting in effects on T cell function including, e.g., pathogenic T cell apoptosis, anergy, and/or exhaustion. In some embodiments, the effect(s) of the methods and compositions of the present invention on T cell function are evaluated by any appropriate method described in the art, e.g., by Keir, et al., 2008.

The methods and compositions of the invention can be used to increase the expression of PD-L1 in a subject. The increased expression of PD-L1 in autoimmune disease target tissues can result in suppression and/or control of autoimmune responses that are involved in the autoimmune disease. In some embodiments, autoimmune responses, e.g., autoreactive T cell responses, are suppressed and/or controlled by interaction of PD-L1 with PD-1 on activated CD4+ and CD8+ T cells, e.g., killer CD8+ T cells. Overexpression of PD-L1 has been described as inducing a T cell switch from pathogenic CD4+T_{H}1 or T_{H}17 effector phenotypes to a T cell regulatory phenotype, for example, a FOXP3+ regulatory T cells (TRE_{G}) phenotype, in vivo, while maintaining T cell receptor antigen specificity (Amarnath et al., 2011, Science Translational Medicine 3(111):1-13, incorporated herein by reference). In some embodiments, the methods and compositions of the invention are used to increase the expression of PD-L1 to induce a T cell switch from pathogenic CD4+ T cells, e.g., T_{H}1 or T_{H}17 cells, to regulatory T cells, e.g., FOXP3+ T cells. In some embodiments, a switch to a Tr1 regulatory phenotype is induced. Tr1 cells express IL-10, but derive classically from exposure of Th1 cells (expressing Tbet transcription factor) upon exposure to IL-27 and other tolerizing stimuli (potentially PD-L1). In some embodiments, the T cell regulatory phenotype is stably maintained long-term. In some embodiments, long-term stability of the T cell regulatory is indicated by the presence of the T_{REG} phenotype for least about 30 days, at least about 35 days, at least about 40 days, at least about 45 days, at least about 50 days, at least about 55 days, at least about 60 days, at least about 65 days, at least about 70 days, at least about 75 days, at least about 80 days, at least about 85 days, at least about 90 days, at least about 95 days, at least about 100 days, at least about 110 days, at least about 120 days, about 30 days to about 90 days, about 40 days to about 90 days, about 50 days to about 90 days, about 60 days to about 90 days, about 70 days to about 90 days, about 30 days to about 100 days, about 40 days to about 100 days, about 50 days to about 100 days, about 60 days to about 100 days, about 70 days to about 100 days, about 80 days to about 100 days, about 30 days to about 120 days, about 40 days to about 120 days, about 50 days to about 120 days, about 60 days to about 120 days, about 70 days to about 120 days, about 80 days to about 120 days, about 30 days to about 120 days, about 40 days to about 120 days, about 50 days to about 120 days, about 60 days to about 120 days, about 70 days to about 120 days, about 80 days to about 120 days, about 30 days to about 150 days, about 40 days to about 150 days, about 50 days to about 150 days, about 60 days to about 150 days, about 70 days to about 150 days, about 80 days to about 150 days, about 30 days to about 150 days, about 40 days to about 150 days, about 50 days to about 150 days, about 60 days to about 150 days, about 70 days to about 150 days, about 80 days to about 150 days, about 30 days to about 200 days, about 40 days to about 200 days, about 50 days to about 200 days, about 60 days to about 200 days, about 70 days to about 200 days, about 80 days to about 200 days, about 30 days to about 200 days, about 40 days to about 200 days, about 50 days to about 200 days, about 60 days to about 200 days, about 70 days to about 200 days, or about 80 days to about 200 days.

In some embodiments, the presence of the regulatory T cell phenotype is determined based on the expression of a T_{R}EG-associated marker or secreted cytokine. In some embodiments, the presence of the regulatory T cell phenotype is determined based on the expression of one or more T_{REG}-associated marker selected from: CD4, CD25, CD39, CD73, CD45RO, CD121a (IL-1R1), CD121b (IL-1R2), CD127low, CD134 (OX40), CD137 (4-1BB), CD152 (CTLA-4), CD357 (GITR/AITR), FOXP3, FR4 (m), GARP, Helios, LAP/TGFβ, and TIGIT; and/or expression of one or more TRE_{G}-associated secreted cytokine selected from: IL-10, IL-35, and TGFβ. In some embodiments, the presence of the T_{REG} phenotype is determined based on the expression of FOXP3. T cell marker expression or cytokine secretion can be evaluated by any method known to those of skill in the art and described in the literature, e.g., by Amarnath, et al., 2011, including, e.g., ELISA, Western Blot assay, flow cytometric assay, or any appropriate multiplex assay. Commercial assays are available for measuring production of cytokines, chemokines, cytokine receptors, and activation markers as called for in the methods of the present invention. For example, multiplex assays for detection of human cytokines and chemokines can be created using the BD™ Cytometric Bead Array Flex Set system (BD Biosciences, San Jose, Calif.).

### Alternative-Intron-Containing Pre-mRNA (AIC Pre-mRNA)

In some embodiments, the methods of the present invention exploit the presence of alternative-intron-containing pre-mRNA (AIC pre-mRNA) transcribed from the CD274 gene and encoding PD-L1 protein, in the cell. Splicing of the identified CD274 AIC pre-mRNA species to produce mature, fully-spliced, CD274 mRNA, is induced using therapeutic agents or antisense oligomers (ASOs) that inhibit splicing of the alternative-introns. The resulting mature CD274 mRNA can be exported to the cytoplasm and translated, thereby increasing the amount of PD-L1 protein in the patient's cells and alleviating symptoms of immune disorders or any disease or disorder associated with deficient amount or activity of PD-L1 protein or induced by a loss-of-function mutation in the CD274 gene.

Table 1 provides a non-limiting list of sequences of CD274 ASOs, useful for increasing production of PD-L1 by targeting a region of a CD274 AIC pre-mRNA. In some embodiments, other ASOs useful for these purposes are identified, using, e.g., methods described herein.

TABLE 1
List of ASOs targeting the CD274 gene

| SEQ ID | | | Genomic coordinate | | |
|---|---|---|---|---|---|
| NO: | ASO# | ID | Chromosome | Start | End |
| 1 | 1 | CD274-EX4 + 68 | chr9 | 5462900 | 5462918 |
| 2 | 2 | CD274-EX4 + 73 | chr9 | 5462905 | 5462923 |
| 3 | 3 | CD274-EX4 + 78 | chr9 | 5462910 | 5462928 |
| 4 | 4 | CD274-EX4 + 83 | chr9 | 5462915 | 5462933 |
| 5 | 5 | CD274-EX4 + 88 | chr9 | 5462920 | 5462938 |
| 6 | 6 | CD274-EX4 + 93 | chr9 | 5462925 | 5462943 |
| 7 | 7 | CD274-EX4 + 98 | chr9 | 5462930 | 5462948 |
| 8 | 8 | CD274-EX4 + 103 | chr9 | 5462935 | 5462953 |
| 9 | 9 | CD274-EX4 + 108 | chr9 | 5462940 | 5462958 |
| 10 | 10 | CD274-EX4 + 113 | chr9 | 5462945 | 5462963 |
| 11 | 11 | CD274-EX4 + 118 | chr9 | 5462950 | 5462968 |
| 12 | 12 | CD274-EX4 + 123 | chr9 | 5462955 | 5462973 |
| 13 | 13 | CD274-EX4 + 128 | chr9 | 5462960 | 5462978 |
| 14 | 14 | CD274-EX4 + 130 | chr9 | 5462962 | 5462980 |
| 15 | 15 | CD274-EX4 + 132 | chr9 | 5462964 | 5462982 |
| 16 | 16 | CD274-EX4 + 134 | chr9 | 5462966 | 5462984 |
| 17 | 17 | CD274-EX4 + 136 | chr9 | 5462968 | 5462986 |
| 18 | 18 | CD274-EX4 + 138 | chr9 | 5462970 | 5462988 |
| 19 | 19 | CD274-EX4 + 143 | chr9 | 5462975 | 5462993 |
| 20 | 20 | CD274-EX4 + 148 | chr9 | 5462980 | 5462998 |
| 21 | 21 | CD274-EX4 + 153 | chr9 | 5462985 | 5463003 |
| 22 | 22 | CD274-EX4 + 158 | chr9 | 5462990 | 5463008 |
| 23 | 23 | CD274-EX4 + 163 | chr9 | 5462995 | 5463013 |
| 24 | 24 | CD274-EX4 + 168 | chr9 | 5463000 | 5463018 |
| 25 | 25 | CD274-EX4 + 173 | chr9 | 5463005 | 5463023 |
| 26 | 26 | CD274-EX4 + 178 | chr9 | 5463010 | 5463028 |
| 27 | 27 | CD274-EX4 + 183 | chr9 | 5463015 | 5463033 |
| 28 | 28 | CD274-EX4 + 188 | chr9 | 5463020 | 5463038 |
| 29 | 29 | CD274-EX4 + 193 | chr9 | 5463025 | 5463043 |
| 30 | 30 | CD274-EX4 + 198 | chr9 | 5463030 | 5463048 |
| 31 | 31 | CD274-EX4 + 203 | chr9 | 5463035 | 5463053 |
| 32 | 32 | CD274-EX4 + 208 | chr9 | 5463040 | 5463058 |
| 33 | 33 | CD274-EX4 + 213 | chr9 | 5463045 | 5463063 |
| 34 | 34 | CD274-EX4 + 218 | chr9 | 5463050 | 5463068 |
| 35 | 35 | CD274-EX4 + 223 | chr9 | 5463055 | 5463073 |
| 36 | 36 | CD274-EX4 + 226 | chr9 | 5463058 | 5463076 |
| 37 | 37 | CD274-EX4 + 228 | chr9 | 5463060 | 5463078 |
| 38 | 38 | CD274-EX4 + 230 | chr9 | 5463062 | 5463080 |
| 39 | 39 | CD274-EX4 + 233 | chr9 | 5463065 | 5463083 |
| 40 | 40 | CD274-EX4 + 238 | chr9 | 5463070 | 5463088 |
| 41 | 41 | CD274-EX4 + 243 | chr9 | 5463075 | 5463093 |
| 42 | 42 | CD274-EX4 + 248 | chr9 | 5463080 | 5463098 |
| 43 | 43 | CD274-EX4 + 253 | chr9 | 5463085 | 5463103 |
| 44 | 44 | CD274-ex4 + 214 | chr9 | 5463046 | 5463064 |
| 45 | 45 | CD274-ex4 + 215 | chr9 | 5463047 | 5463065 |
| 46 | 46 | CD274-ex4 + 216 | chr9 | 5463048 | 5463066 |
| 47 | 47 | CD274-ex4 + 217 | chr9 | 5463049 | 5463067 |
| 48 | 48 | CD274-ex4 + 219 | chr9 | 5463051 | 5463069 |
| 49 | 49 | CD274-ex4 + 220 | chr9 | 5463052 | 5463070 |
| 50 | 50 | CD274-ex4 + 221 | chr9 | 5463053 | 5463071 |
| 51 | 51 | CD274-ex4 + 222 | chr9 | 5463054 | 5463072 |
| 52 | 52 | CD274-ex4 + 239 | chr9 | 5463071 | 5463089 |
| 53 | 53 | CD274-ex4 + 240 | chr9 | 5463072 | 5463090 |
| 54 | 54 | CD274-ex4 + 241 | chr9 | 5463073 | 5463091 |
| 55 | 55 | CD274-ex4 + 242 | chr9 | 5463074 | 5463092 |
| 56 | 56 | CD274-ex4 + 244 | chr9 | 5463076 | 5463094 |
| 57 | 57 | CD274-ex4 + 245 | chr9 | 5463077 | 5463095 |
| 58 | 58 | CD274-ex4 + 246 | chr9 | 5463078 | 5463096 |
| 59 | 59 | CD274-ex4 + 247 | chr9 | 5463079 | 5463097 |
| 60 | 60 | CD274-ex4 + 249 | chr9 | 5463081 | 5463099 |
| 61 | 61 | CD274-ex4 + 250 | chr9 | 5463082 | 5463100 |
| 62 | 62 | CD274-ex4 + 251 | chr9 | 5463083 | 5463101 |
| 63 | 63 | CD274-ex4 + 252 | chr9 | 5463084 | 5463102 |
| 64 | 64 | CD274-ex4 + 254 | chr9 | 5463086 | 5463104 |
| 65 | 65 | CD274-ex4 + 255 | chr9 | 5463087 | 5463105 |
| 66 | 66 | CD274-ex4 + 256 | chr9 | 5463088 | 5463106 |
| 67 | 67 | CD274-ex4 + 257 | chr9 | 5463089 | 5463107 |

In some embodiments, the targeted region of the CD274 AIC pre-mRNA is in exon 4. The CD274 intron numbering used herein corresponds to the mRNA sequence at NM_014143.3. In some embodiments, hybridization of an ASO to the targeted region of the AIC pre-mRNA results in inhibition of splicing at the alternative splice site (5′ splice site or 3′ splice site) in the alternative-intron in exon 4 and subsequently increases PD-L1 protein production. It is understood that the intron numbering may change in reference to a different CD274 isoform sequence. One of skill in the art can determine the corresponding intron number in any CD274 isoform based on an intron sequence provided herein or using the intron number provided in reference to the mRNA sequence at NM_014143.3. One of skill in the art also can determine the sequences of flanking exons in any CD274 isoform for targeting using the methods of the invention, based on an intron sequence provided herein or using the intron number provided in reference to the mRNA sequence at NM_014143.3. In some embodiments, the methods and compositions of the present invention are used to increase the expression of any known CD274 isoform.

In some embodiments, the methods of the present invention exploit the presence of alternative-intron-containing pre-mRNA (AIC pre-mRNA) transcribed from the ARHGAP23 gene and encoding Rho GTPase Activating Protein 23 protein, in the cell. Splicing of the identified ARHGAP23 AIC pre-mRNA species to produce mature, fully-spliced, ARHGAP23 mRNA, is induced using therapeutic agents or antisense oligomers (ASOs) that inhibit splicing of the alternative-introns. The resulting mature ARHGAP23 mRNA can be exported to the cytoplasm and translated, thereby increasing the amount of Rho GTPase Activating Protein 23 protein in the patient's cells and alleviating symptoms of hormonal disorders or any disease or disorder associated with deficient amount or activity of Rho GTPase Activating Protein 23 protein or induced by a loss-of-function mutation in the ARHGAP23 gene.

In some embodiments, the methods of the present invention exploit the presence of alternative-intron-containing pre-mRNA (AIC pre-mRNA) transcribed from the BRD1 gene and encoding the Bromodomain Containing 1 protein, in the cell. Splicing of the identified BRD1 AIC pre-mRNA species to produce mature, fully-spliced, BRD1 mRNA, is induced using therapeutic agents or antisense oligomers (ASOs) that inhibit splicing of the alternative-introns. The resulting mature BRD1 mRNA can be exported to the cytoplasm and translated, thereby increasing the amount of Bromodomain Containing 1 protein in the patient's cells and alleviating symptoms of immune disorders or any disease or disorder associated with deficient amount or activity of Bromodomain Containing 1 protein or induced by a loss-of-function mutation in the BRD1 gene.

In some embodiments, the methods of the present invention exploit the presence of alternative-intron-containing pre-mRNA (AIC pre-mRNA) transcribed from the DCHS1 gene and encoding Protocadherin-16 protein, in the cell. Splicing of the identified DCHS1 AIC pre-mRNA species to produce mature, fully-spliced, DCHS1 mRNA, is induced using therapeutic agents or antisense oligomers (ASOs) that inhibit splicing of the alternative-introns. The resulting mature DCHS1 mRNA can be exported to the cytoplasm and translated, thereby increasing the amount of Protocadherin-16 protein in the patient's cells and alleviating symptoms of cardiac disorders or any disease or disorder associated with deficient amount or activity of Protocadherin-16 protein or induced by a loss-of-function mutation in the DCHS1 gene.

In some embodiments, the methods of the present invention exploit the presence of alternative-intron-containing pre-mRNA (AIC pre-mRNA) transcribed from the EPB41L2 gene and encoding Band 4.1-like protein 2 protein, in the cell. Splicing of the identified EPB41L2 AIC pre-mRNA species to produce mature, fully-spliced, EPB41L2 mRNA, is induced using therapeutic agents or antisense oligomers (ASOs) that inhibit splicing of the alternative-introns. The resulting mature EPB41L2 mRNA can be exported to the cytoplasm and translated, thereby increasing the amount of Band 4.1-like protein 2 protein in the patient's cells and alleviating symptoms of liver related disorders or any disease or disorder associated with deficient amount or activity of Band 4.1-like protein 2 protein or induced by a loss-of-function mutation in the EPB41L2 gene.

In some embodiments, the methods of the present invention exploit the presence of alternative-intron-containing pre-mRNA (AIC pre-mRNA) transcribed from the GPX8 gene and encoding glutathione peroxidase 8 protein, in the cell. Splicing of the identified GPX8 AIC pre-mRNA species to produce mature, fully-spliced, GPX8 mRNA, is induced using therapeutic agents or antisense oligomers (ASOs) that inhibit splicing of the alternative-introns. The resulting mature GPX8 mRNA can be exported to the cytoplasm and translated, thereby increasing the amount of glutathione peroxidase 8 protein in the patient's cells and alleviating symptoms of liver related disorders or any disease or disorder associated with deficient amount or activity of glutathione peroxidase 8 protein or induced by a loss-of-function mutation in the GPX8 gene.

In some embodiments, the methods of the present invention exploit the presence of alternative-intron-containing pre-mRNA (AIC pre-mRNA) transcribed from the HIVEP3 gene and encoding Human immunodeficiency virus type I enhancer-binding protein 3 protein, in the cell. Splicing of the identified HIVEP3 AIC pre-mRNA species to produce mature, fully-spliced, HIVEP3 mRNA, is induced using therapeutic agents or antisense oligomers (ASOs) that inhibit splicing of the alternative-introns. The resulting mature HIVEP3 mRNA can be exported to the cytoplasm and translated, thereby increasing the amount of Human immunodeficiency virus type I enhancer-binding protein 3 protein in the patient's cells and alleviating symptoms of immune disorders or any disease or disorder associated with deficient amount or activity of Human immunodeficiency virus type I enhancer-binding protein 3 protein or induced by a loss-of-function mutation in the HIVEP3 gene.

In some embodiments, the methods of the present invention exploit the presence of alternative-intron-containing pre-mRNA (AIC pre-mRNA) transcribed from the INVS gene and encoding Inversin protein, in the cell. Splicing of the identified INVS AIC pre-mRNA species to produce mature, fully-spliced, INVS mRNA, is induced using therapeutic agents or antisense oligomers (ASOs) that inhibit splicing of the alternative-introns. The resulting mature INVS mRNA can be exported to the cytoplasm and translated, thereby increasing the amount of Inversin protein in the patient's cells and alleviating symptoms of immune disorders or any disease or disorder associated with deficient amount or activity of Inversin protein or induced by a loss-of-function mutation in the INVS gene.

In some embodiments, the methods of the present invention exploit the presence of alternative-intron-containing pre-mRNA (AIC pre-mRNA) transcribed from the KIAA0319 gene and encoding Dyslexia-associated protein KIAA0319 protein, in the cell. Splicing of the identified KIAA0319 AIC pre-mRNA species to produce mature, fully-spliced, KIAA0319 mRNA, is induced using therapeutic agents or antisense oligomers (ASOs) that inhibit splicing of the alternative-introns. The resulting mature KIAA0319 mRNA can be exported to the cytoplasm and translated, thereby increasing the amount of Dyslexia-associated protein KIAA0319 protein in the patient's cells and alleviating symptoms of developmental disorders or any disease or disorder associated with deficient amount or activity of Dyslexia-associated protein KIAA0319 protein or induced by a loss-of-function mutation in the KIAA0319 gene.

In some embodiments, the methods of the present invention exploit the presence of alternative-intron-containing pre-mRNA (AIC pre-mRNA) transcribed from the NAIP gene and encoding NLR Family Apoptosis Inhibitory protein, in the cell. Splicing of the identified NAIP AIC pre-mRNA species to produce mature, fully-spliced, NAIP mRNA, is induced using therapeutic agents or antisense oligomers (ASOs) that inhibit splicing of the alternative-introns. The resulting mature NAIP mRNA can be exported to the cytoplasm and translated, thereby increasing the amount of NLR Family Apoptosis Inhibitory protein in the patient's cells and alleviating symptoms of muscle related disorders or any disease or disorder associated with deficient amount or activity of NLR Family Apoptosis Inhibitory protein or induced by a loss-of-function mutation in the NAIP gene.

In some embodiments, the methods of the present invention exploit the presence of alternative-intron-containing pre-mRNA (AIC pre-mRNA) transcribed from the PTCH2 gene and encoding Protein patched homolog 2 protein, in the cell. Splicing of the identified PTCH2 AIC pre-mRNA species to produce mature, fully-spliced, PTCH2 mRNA, is induced using therapeutic agents or antisense oligomers (ASOs) that inhibit splicing of the alternative-introns. The resulting mature PTCH2 mRNA can be exported to the cytoplasm and translated, thereby increasing the amount of Protein patched homolog 2 protein in the patient's cells and alleviating symptoms of immune disorders or any disease or disorder associated with deficient amount or activity of Protein patched homolog 2 protein or induced by a loss-of-function mutation in the PTCH2 gene.

In some embodiments, the methods of the present invention exploit the presence of alternative-intron-containing pre-mRNA (AIC pre-mRNA) transcribed from the PTPRZ1 gene and encoding Protein Tyrosine Phosphatase Receptor Type Z1 protein, in the cell. Splicing of the identified PTPRZ1 AIC pre-mRNA species to produce mature, fully-spliced, PTPRZ1 mRNA, is induced using therapeutic agents or antisense oligomers (ASOs) that inhibit splicing of the alternative-introns. The resulting mature PTPRZ1 mRNA can be exported to the cytoplasm and translated, thereby increasing the amount of Protein Tyrosine Phosphatase Receptor Type Z1 protein in the patient's cells and alleviating symptoms of mental disorders or any disease or disorder associated with deficient amount or activity of Protein Tyrosine Phosphatase Receptor Type Z1 or induced by a loss-of-function mutation in the PTPRZ1 gene.

In some embodiments, the methods of the present invention exploit the presence of alternative-intron-containing pre-mRNA (AIC pre-mRNA) transcribed from the SONgene and encoding SON protein, in the cell. Splicing of the identified SON AIC pre-mRNA species to produce mature, fully-spliced, SON mRNA, is induced using therapeutic agents or antisense oligomers (ASOs) that inhibit splicing of the alternative-introns. The resulting mature SON mRNA can be exported to the cytoplasm and translated, thereby increasing the amount of SON protein in the patient's cells and alleviating symptoms of immune disorders or any disease or disorder associated with deficient amount or activity of SON protein or induced by a loss-of-function mutation in the SON gene.

In some embodiments, the methods of the present invention exploit the presence of alternative-intron-containing pre-mRNA (AIC pre-mRNA) transcribed from the ZCCHC2 gene and encoding Zinc finger CCHC domain-containing protein 2 protein, in the cell. Splicing of the identified ZCCHC2 AIC pre-mRNA species to produce mature, fully-spliced, ZCCHC2 mRNA, is induced using therapeutic agents or antisense oligomers (ASOs) that inhibit splicing of the alternative-introns. The resulting mature ZCCHC2 mRNA can be exported to the cytoplasm and translated, thereby increasing the amount of Zinc finger CCHC domain-containing protein 2 protein in the patient's cells and alleviating symptoms of immune disorders or any disease or disorder associated with deficient amount or activity of Zinc finger CCHC domain-containing protein 2 protein or induced by a loss-of-function mutation in the ZCCHC2 gene.

Table 3 provides a non-limiting list of sequences of ASOs, useful for increasing production of proteins encoded by ARHGAP23, BRD1, DCHS1, EPB41L2, GPX8, HIVEP3, INVS, KIAA0319, NAIP, PD-L1, PTCH2, PTPRZ1, SON, ZCCHC2 by targeting a region of the AIC pre-mRNA. In some embodiments, other ASOs useful for these purposes are identified, using, e.g., methods described herein.

### Protein Expression

In some embodiments, the methods described herein are used to increase the production of a protein in a subject in need thereof. In some embodiments, the methods described herein are used to increase the production of a functional protein in a subject in need thereof. As used herein, the term “functional” refers to the amount of activity or function of a protein that is necessary to eliminate any one or more symptoms of a treated condition, e.g., a mental disorder caused by a genetic defect in BRD1 gene. In some embodiments, the methods are used to increase the production of a fully functional protein or RNA. In some embodiments, the methods are used to increase the production of a partially functional protein or RNA. As used herein, the term “partially functional” refers to any amount of activity or function of the protein that is less than the amount of activity or function that is necessary to eliminate or prevent any one or more symptoms of a disease or condition. In some embodiments, a partially functional protein or RNA will have at least 10%, at least 20%, at least 30%, at least 40%, at least 50%, at least 60%, at least 70%, at least 75%, at least 80%, 85%, at least 90%, or at least 95% less activity relative to the fully functional protein or RNA. In some embodiments, the target protein and the RIC pre-mRNA are encoded by a gene selected from: ARHGAP23, BRD1, DCHS1, EPB41L2, GPX8, HIVEP3, INVS, KIAA0319, NAIP, PD-L1, PTCH2, PTPRZ1, SON, ZCCHC2.

In some embodiments, the methods described herein are used to increase the production of a PD-L1 protein in a subject in need thereof. In some embodiments, the methods described herein are used to increase the production of a functional PD-L1 protein in a subject in need thereof. As used herein, the term “functional” refers to the amount of activity or function of a PD-L1 protein that is necessary to eliminate any one or more symptoms of a treated condition, e.g., an immune disorder caused by a genetic defect in PD-L1. In some embodiments, the methods are used to increase the production of a fully functional PD-L1 protein or RNA. In some embodiments, the methods are used to increase the production of a partially functional PD-L1 protein or RNA. In some embodiments, the method is a method of increasing the expression of the target protein by cells of a subject having an AIC pre-mRNA encoding the target protein, wherein the subject has an immune disease or disorder caused by a deficient amount or activity of the target protein. In some embodiments, the deficient amount of the target protein is caused by haploinsufficiency of the target protein. In some embodiments, the disease or disorder is associated with haploinsufficiency of a gene encoding the target protein. In such embodiments, the subject has a first allele encoding a functional the target protein, and a second allele from which the target protein is not produced. In such embodiments, the subject has a first allele encoding a functional target protein, and a second allele from which the target protein is produced at a reduced level. In another such embodiment, the subject has a first allele encoding a functional the target protein, and a second allele encoding a nonfunctional target protein. In another such embodiment, the subject has a first allele encoding a functional the target protein, and a second allele encoding a partially functional the target protein. In any of these embodiments, the ASO binds to a targeted region of the AIC pre-mRNA transcribed from both alleles, the first allele (encoding the functional target protein) and the second allele (from which the target protein is not produced or the target protein is produced at reduced level or the target protein is non-functional), thereby inhibiting splicing of the alternative-intron from the AIC pre-mRNA, and causing an increase in the level of mature mRNA from the first allele encoding the functional target protein, and an increase in the expression of the target protein in the cells of the subject. In some embodiments, the target protein and the RIC pre-mRNA are encoded by a gene selected from: ARHGAP23, BRD1, DCHS1, EPB41L2, GPX8, HIVEP3, INVS, KIAA0319, NAIP, CD274, PTCH2, PTPRZ1, SON, ZCCHC2.

In some embodiments, the method is a method of increasing the expression of PD-L1 protein by cells of a subject having an AIC pre-mRNA encoding PD-L1 protein, wherein the subject has an immune disease or disorder caused by a deficient amount or activity of PD-L1 protein. In some embodiments, the deficient amount of PD-L1 protein is caused by haploinsufficiency of PD-L1 protein. In some embodiments, the disease or disorder is associated with haploinsufficiency of a gene encoding PD-L1 protein. In such embodiments, the subject has a first allele encoding a functional PD-L1 protein, and a second allele from which PD-L1 protein is not produced. In such embodiments, the subject has a first allele encoding a functional PD-L1 protein, and a second allele from which PD-L1 protein is produced at a reduced level. In another such embodiment, the subject has a first allele encoding a functional PD-L1 protein, and a second allele encoding a nonfunctional PD-L1 protein. In another such embodiment, the subject has a first allele encoding a functional PD-L1 protein, and a second allele encoding a partially functional PD-L1 protein. In any of these embodiments, the ASO binds to a targeted region of the AIC pre-mRNA transcribed from the first allele (encoding functional PD-L1 protein), thereby inhibiting splicing of the alternative-intron from the AIC pre-mRNA, and causing an increase in the level of mature mRNA encoding functional PD-L1 protein, and an increase in the expression of PD-L1 protein in the cells of the subject.

In some embodiments, the subject has a first allele encoding a functional the target protein, and a second allele encoding a partially functional the target protein, and the ASO binds to a targeted region of the AIC pre-mRNA transcribed from the first allele (encoding functional the target protein) or a targeted region of the AIC pre-mRNA transcribed from the second allele (encoding partially functional the target protein), thereby inhibiting splicing of the alternative-intron from the AIC pre-mRNA, and causing an increase in the level of mature mRNA encoding the target protein, and an increase in the expression of functional or partially functional the target protein in the cells of the subject. In some embodiments, the target protein and the RIC pre-mRNA are encoded by a gene selected from: ARHGAP23, BRD1, DCHS1, EPB41L2, GPX8, HIVEP3, INVS, KIAA0319, NAIP, PD-L1, PTCH2, PTPRZ1, SON, ZCCHC2.

In some embodiments, the subject has a first allele encoding a functional PD-L1 protein, and a second allele encoding a partially functional PD-L1 protein, and the ASO binds to a targeted region of the AIC pre-mRNA transcribed from the first allele (encoding functional PD-L1 protein) or a targeted region of the AIC pre-mRNA transcribed from the second allele (encoding partially functional PD-L1 protein), thereby inhibiting splicing of the alternative-intron from the AIC pre-mRNA, and causing an increase in the level of mature mRNA encoding PD-L1 protein, and an increase in the expression of functional or partially functional PD-L1 protein in the cells of the subject.

In related embodiments, the method is a method of using an ASO to increase the expression of a protein or functional RNA. In some embodiments, the target protein and the RIC pre-mRNA are encoded by a gene selected from: ARHGAP23, BRD1, DCHS1, EPB41L2, GPX8, HIVEP3, INVS, KIAA0319, NAIP, CD274, PTCH2, PTPRZ1, SON, ZCCHC2. In some embodiments, an ASO is used to increase the expression of PD-L1 protein in cells of a subject having an AIC pre-mRNA encoding PD-L1 protein, wherein the subject has a deficiency in the amount or function of a PD-L1 protein.

In some embodiments, the AIC pre-mRNA transcript that encodes the protein that is causative of the disease or condition is targeted by the ASOs described herein. In some embodiments, an AIC pre-mRNA transcript that encodes a protein that is not causative of the disease is targeted by the ASOs. For example, a disease that is the result of a mutation or deficiency of a first protein in a particular pathway may be ameliorated by targeting an AIC pre-mRNA that encodes a second protein, thereby increasing production of the second protein. In some embodiments, the function of the second protein is able to compensate for the mutation or deficiency of the first protein (which is causative of the disease or condition).

In some embodiments, the subject has:

a. a first mutant allele from which
i) protein is produced at a reduced level compared to production from a wild-type allele,
ii) protein is produced in a form having reduced function compared to an equivalent wild-type protein, or
iii) protein or functional RNA is not produced; and

b. a second mutant allele from which
i) protein is produced at a reduced level compared to production from a wild-type allele,
ii) protein is produced in a form having reduced function compared to an equivalent wild-type protein, and
   wherein the AIC pre-mRNA is transcribed from the first allele and the second allele and wherein the target protein or the functional RNA are encoded by a gene selected from: ARHGAP23, BRD1, DCHS1, EPB41L2, GPX8, HIVEP3, INVS, KIAA0319, NAIP, CD274, PTCH2, PTPRZ1, SON, ZCCHC2. In these embodiments, the ASO binds to a targeted region of the AIC pre-mRNA transcribed from the first allele and the second allele, thereby inhibiting splicing of the alternative-intron from the AIC pre-mRNA, and causing an increase in the level of mRNA encoding the protein and an increase in the expression of the target protein or target functional RNA in the cells of the subject. In these embodiments, the protein or functional RNA having an increase in expression level resulting from inhibiting splicing of the alternative-intron from the AIC pre-mRNA is either in a form having reduced function compared to the equivalent wild-type protein (partially-functional), or having full function compared to the equivalent wild-type PD-L1 protein (fully-functional).

In some embodiments, the level of processed mRNA encoding the target protein is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell, e.g., one that is not treated with the ASO or one that is treated with an ASO that does not bind to the targeted region of the target AIC pre-mRNA; wherein the target protein is encoded by a gene selected from: ARHGAP23, BRD1, DCHS1, EPB41L2, GPX8, HIVEP3, INVS, KIAA0319, NAIP, CD274, PTCH2, PTPRZ1, SON, ZCCHC2.

In some embodiments, a subject treated using the methods of the invention expresses a partially functional the target protein from one allele, wherein the partially functional the target protein is caused by a frameshift mutation, a nonsense mutation, a missense mutation, a splicing mutation, or a partial gene deletion. In some embodiments, a subject treated using the methods of the invention expresses a nonfunctional the target protein from one allele, wherein the nonfunctional the target protein is caused by a frameshift mutation, a nonsense mutation, a missense mutation, a splicing mutation, or a partial gene deletion, in one allele. In some embodiments, a subject treated using the methods of the invention has a target whole gene deletion, in one allele. In some embodiments, the target protein is encoded by a gene selected from: ARHGAP23, BRD1, DCHS1, EPB41L2, GPX8, HIVEP3, INVS, KIAA0319, NAIP, CD274, PTCH2, PTPRZ1, SON, ZCCHC2.

### Increasing Protein Expression

As described above, in some embodiments, the methods of the invention are used to increase expression of the target protein. The target protein may be encoded by a gene selected from: ARHGAP23, BRD1, DCHS1, EPB41L2, GPX8, HIVEP3, INVS, KIAA0319, NAIP, CD274, PTCH2, PTPRZ1, SON, ZCCHC2. In these embodiments, an alternative-intron pre-mRNA (AIC pre-mRNA) encoding the target protein is present in the nucleus of a cell. Cells having a the target AIC pre-mRNA that comprises an alternative-intron, a first portion of an exon flanking a 5′ splice site of the alternative-intron, and a second portion of the exon flanking a 3′ splice site of the alternative-intron, encoding the target protein, are contacted with antisense oligomers (ASOs) that are complementary to a targeted region of the AIC pre-mRNA. Hybridization of the ASOs to the targeted region of the AIC pre-mRNA results in inhibition of splicing of the alternative-intron and subsequently increases target protein production.

The terms “pre-mRNA,” and “pre-mRNA transcript” may be used interchangeably and refer to any pre-mRNA species that contains at least one intron. In some embodiments, pre-mRNA or pre-mRNA transcripts comprise a 5′-7-methylguanosine cap and/or a poly-A tail. In some embodiments, pre-mRNA or pre-mRNA transcripts comprise both a 5′-7-methylguanosine cap and a poly-A tail. In some embodiments, the pre-mRNA transcript does not comprise a 5′-7-methylguanosine cap and/or a poly-A tail. A pre-mRNA transcript is a non-productive messenger RNA (mRNA) molecule if it is not translated into a protein (or transported into the cytoplasm from the nucleus).

As used herein, a “alternative-intron-containing pre-mRNA” (“AIC pre-mRNA”) is a pre-mRNA transcript that contains at least one alternative-intron. The AIC pre-mRNA contains an alternative-intron, a first portion of an exon flanking a 5′ splice site of the alternative-intron, and a second portion of the exon flanking a 3′ splice site of the alternative-intron and encodes the target protein. A “AIC pre-mRNA encoding a target protein” is understood to encode the target protein when fully spliced. A “alternative-intron” is a region or sequence between a 5′ alternative splice site and a 3′ alternative splice site within an exon in a pre-mRNA transcript that can be spliced out when the pre-mRNA is processed to produce an mRNA (the processed mRNA is missing a portion of the exon).

In some embodiments, the therapeutic agent or the ASO is at least 70%, at least 75%, at least 80%, at least 85%, at least 90%, at least 95%, at least 98%, at least 99%, or 100%, complementary to the targeted region of the AIC pre-mRNA encoding the target protein. In some embodiments, at least a portion of the targeted region of the target AIC pre-mRNA is within an intron upstream of the first portion of the exon. In some embodiments, at least a portion of the targeted region of the target AIC pre-mRNA is within an intron downstream of the second portion of the exon. In some embodiments, at least a portion of the targeted region of the target AIC pre-mRNA is within the alternative-intron. In some embodiments, at least a portion of the targeted region of the target AIC pre-mRNA is within the first portion of the exon. In some embodiments, at least a portion of the targeted region of the target AIC pre-mRNA is within the second portion of the exon. In some embodiments, at least a portion of the targeted region of the target AIC pre-mRNA overlaps with a junction of the first portion of the exon and the alternative-intron. In some embodiments, at least a portion of the targeted region of the target AIC pre-mRNA overlaps with a junction of the alternative-intron and the second portion of the exon. As used to identify the location of a region or sequence, “within” is understood to include the residues at the positions recited. For example, a region +6 to +100 includes the residues at positions+6 and +100. In some embodiments, the target AIC pre-mRNA is encoded by a gene selected from: ARHGAP23, BRD1, DCHS1, EPB41L2, GPX8, HIVEP3, INVS, KIAA0319, NAIP, CD274, PTCH2, PTPRZ1, SON, ZCCHC2.

In some embodiments, a mature mRNA encoding the target protein is thereby produced. In some embodiments, the target protein produced is fully functional the target protein. In some embodiments, the target RNA produced is functional the target RNA. In some embodiments, the target RNA produced is fully functional the target RNA. The terms “mature mRNA,” “fully-spliced mRNA,” and functional RNA” are used interchangeably herein to describe a fully processed mRNA encoding a target protein (e.g., mRNA that is exported from the nucleus into the cytoplasm and translated into target protein) or a fully processed functional RNA. The term “productive mRNA,” also can be used to describe a fully processed mRNA encoding a target protein.

As used herein, the term “comprise” or variations thereof such as “comprises” or “comprising” are to be read to indicate the inclusion of any recited feature (e.g. in the case of an ASO, a defined nucleobase sequence) but not the exclusion of any other features. Thus, as used herein, the term “comprising” is inclusive and does not exclude additional, unrecited features (e.g. in the case of an ASO, the presence of additional, unrecited nucleobases).

In some embodiments of any of the methods and compositions provided herein, “comprising” may be replaced with “consisting essentially of” or “consisting of.” The phrase “consisting essentially of” is used herein to require the specified feature(s) (e.g., nucleobase sequence) as well as those which do not materially affect the character or function of the claimed invention. As used herein, the term “consisting” is used to indicate the presence of the recited feature (e.g., nucleobase sequence) alone (so that in the case of an antisense oligomer consisting of a specified nucleobase sequence, the presence of additional, unrecited nucleobases is excluded).

As used herein, the “wild-type sequence” refers to the nucleotide sequence for the target gene in the published reference genome deposited in the NCBI repository of biological and scientific information (operated by National Center for Biotechnology Information, National Library of Medicine, 8600 Rockville Pike, Bethesda, Md. USA 20894). As used herein, the “wild-type sequence” refers to the canonical sequence available at NCBI Gene ID 29126. Also used herein, a nucleotide position denoted with an “e” indicates the nucleotide is present in the sequence of an exon (e.g., the exon flanking the 5′ splice site or the exon flanking the 3′ splice site).

The methods involve contacting cells with a therapeutic agent or an ASO that is complementary to a region of a pre-mRNA encoding the target protein, resulting in increased expression of the target. As used herein, “contacting” or administering to cells refers to any method of providing an ASO in immediate proximity with the cells such that the ASO and the cells interact. A cell that is contacted with an ASO will take up or transport the ASO into the cell. The method involves contacting a condition- or disease-associated or condition- or disease-relevant cell with any of the ASOs described herein. In some embodiments, the ASO may be further modified or attached (e.g., covalently attached) to another molecule to target the ASO to a cell type, enhance contact between the ASO and the condition or disease-associated or condition or disease-relevant cell, or enhance uptake of the ASO.

As used herein, the term “increasing protein production” or “increasing expression of a target protein” means enhancing the amount of protein that is translated from an mRNA in a cell. A “target protein” may be any protein for which increased expression/production is desired.

In some embodiments, contacting a cell that expresses the target AIC pre-mRNA with an ASO that is complementary to a targeted region of the target AIC pre-mRNA transcript results in a measurable increase in the amount of the target protein (e.g., a target protein) encoded by the pre-mRNA. Methods of measuring or detecting production of a protein will be evident to one of skill in the art and include any known method, for example, Western blotting, flow cytometry, immunofluorescence microscopy, and ELISA.

In some embodiments, the therapeutic agent, or ASO increases the expression of the target protein in the cell. In some embodiments, contacting cells with an ASO that is complementary to a targeted region of a the target AIC pre-mRNA transcript results in an increase in the amount of the target protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the expression level of the target protein produced by the cell to which the ASO was contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the expression level of the target protein produced by an control compound. A control compound can be, for example, an oligonucleotide that is not complementary to the targeted region of the AIC pre-mRNA.

In some embodiments, the therapeutic agent, or ASO increases the level of processed mRNA encoding the target protein or mature mRNA encoding the target protein in the cell. In some embodiments, contacting cells with an ASO that is complementary to a targeted region of the target AIC pre-mRNA transcript results in an increased level of processed mRNA encoding the target, including the mature mRNA encoding the target protein. In some embodiments, the level of processed mRNA encoding the target protein, or the mature mRNA encoding the target protein, is increased by at least 10, 20, 30, 40, 50, 60, 80, 100, 150, 200, 250, 300, 350, 400, 450, 500, or 1000%, compared to the level of processed mRNA encoding the target protein in a cell in the absence of the ASO/absence of treatment. In some embodiments, the level of processed mRNA encoding the target protein, or the mature mRNA encoding the target protein produced in the cell to which the ASO was contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the level of processed mRNA or mature RNA in an untreated cell, e.g., an untreated cell or a cell treated with a control compound. A control compound can be, for example, an oligonucleotide that is not complementary to the targeted region of the target AIC pre-mRNA.

### Antisense Oligomers (ASOs)

One aspect of the present disclosure is methods and compositions comprising therapeutic agents that modulate (e.g., inhibit or enhance) splicing by binding to a targeted region of a AIC pre-mRNA. In some embodiments, the present disclosure includes methods and compositions comprising antisense oligomers (ASOs) that modulate (e.g., inhibit or enhance) splicing by binding to a targeted region of a AIC pre-mRNA. As used herein, the terms “ASO,” “antisense oligomer,” and “antisense oligonucleotide” are used interchangeably and refer to an oligomer such as a polynucleotide, comprising nucleobases that hybridizes to a target nucleic acid (e.g., a CD274 AIC pre-mRNA) sequence by Watson-Crick base pairing or wobble base pairing (G-U). The ASO may have exact sequence complementary to the target sequence or near complementarity (e.g., sufficient complementarity to bind the target sequence and enhancing splicing at a splice site). ASOs are designed so that they bind (hybridize) to a target nucleic acid (e.g., a targeted region of a pre-mRNA transcript) and remain hybridized under physiological conditions. Typically, if they hybridize to a site other than the intended (targeted) nucleic acid sequence, they hybridize to a limited number of sequences that are not a target nucleic acid (to a few sites other than a target nucleic acid). Design of an ASO can take into consideration the occurrence of the nucleic acid sequence of the targeted region of the pre-mRNA transcript or a sufficiently similar nucleic acid sequence in other locations in the genome or cellular pre-mRNA or transcriptome, such that the likelihood the ASO will bind other sites and cause “off-target” effects is limited. Any ASOs known in the art, for example in PCT Application No. PCT/US2014/054151, published as WO 2015/035091, titled “Reducing Nonsense-Mediated mRNA Decay,” can be used to practice the methods described herein.

In some embodiments, ASOs “specifically hybridize” to or are “specific” to a target nucleic acid or a targeted region of a AIC pre-mRNA. Typically, such hybridization occurs with a Tm substantially greater than 37° C., preferably at least 50° C., and typically between 60° C. to approximately 90° C. Such hybridization preferably corresponds to stringent hybridization conditions. At a given ionic strength and pH, the Tm is the temperature at which 50% of a target sequence hybridizes to a complementary oligonucleotide.

Oligomers, such as oligonucleotides, are “complementary” to one another when hybridization occurs in an antiparallel configuration between two single-stranded polynucleotides. A double-stranded polynucleotide can be “complementary” to another polynucleotide, if hybridization can occur between one of the strands of the first polynucleotide and the second. Complementarity (the degree to which one polynucleotide is complementary with another) is quantifiable in terms of the proportion (e.g., the percentage) of bases in opposing strands that are expected to form hydrogen bonds with each other, according to generally accepted base-pairing rules. The sequence of an ASO can be 100% complementary to that of its target nucleic acid to hybridize; however, the sequence of an ASO needs not be 100% complementary to that of its target nucleic acid to hybridize. In certain embodiments, ASOs can comprise at least 70%, at least 75%, at least 80%, at least 85%, at least 90%, at least 95%, at least 96%, at least 97%, at least 98%, or at least 99% sequence complementarity to a target region within the target nucleic acid sequence to which they are targeted. For example, an ASO in which 18 of 20 nucleobases of the oligomeric compound are complementary to a target region, and would therefore specifically hybridize, would represent 90 percent complementarity. In this example, the remaining non-complementary nucleobases may be clustered together or interspersed with complementary nucleobases and need not be contiguous to each other or to complementary nucleobases. Percent complementarity of an ASO with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

An ASO needs not hybridize to all nucleobases in a target sequence and the nucleobases to which it does hybridize may be contiguous or noncontiguous. ASOs may hybridize over one or more segments of a pre-mRNA transcript, such that intervening or adjacent segments are not involved in the hybridization event (e.g., a loop structure or hairpin structure may be formed). In certain embodiments, an ASO hybridizes to noncontiguous nucleobases in a target pre-mRNA transcript. For example, an ASO can hybridize to nucleobases in a pre-mRNA transcript that are separated by one or more nucleobase(s) to which the ASO does not hybridize.

The ASOs described herein comprise nucleobases that are complementary to nucleobases present in a target region of a AIC pre-mRNA. The term ASO embodies oligonucleotides and any other oligomeric molecule that comprises nucleobases capable of hybridizing to a complementary nucleobase on a target mRNA but does not comprise a sugar moiety, such as a peptide nucleic acid (PNA). The ASOs may comprise naturally-occurring nucleotides, nucleotide analogs, modified nucleotides, or any combination of two or three of the preceding. The term “naturally occurring nucleotides” includes deoxyribonucleotides and ribonucleotides. The term “modified nucleotides” includes nucleotides with modified or substituted sugar groups and/or having a modified backbone. In some embodiments, all of the nucleotides of the ASO are modified nucleotides. Chemical modifications of ASOs or components of ASOs that are compatible with the methods and compositions described herein will be evident to one of skill in the art and can be found, for example, in U.S. Pat. No. 8,258,109 B2, U.S. Pat. No. 5,656,612, U.S. Patent Publication No. 2012/0190728, and Dias and Stein, Mol. Cancer Ther. 2002, 1, 347-355, herein incorporated by reference in their entirety.

The nucleobase of an ASO may be any naturally occurring, unmodified nucleobase such as adenine, guanine, cytosine, thymine, and uracil, or any synthetic or modified nucleobase that is sufficiently similar to an unmodified nucleobase such that it is capable of hydrogen bonding with a nucleobase present on a target pre-mRNA. Examples of modified nucleobases include, without limitation, hypoxanthine, xanthine, 7-methylguanine, 5, 6-dihydrouracil, 5-methylcytosine, and 5-hydroxymethoylcytosine.

The ASOs described herein also comprise a backbone structure that connects the components of an oligomer. The term “backbone structure” and “oligomer linkages” may be used interchangeably and refer to the connection between monomers of the ASO. In naturally occurring oligonucleotides, the backbone comprises a 3′-5′ phosphodiester linkage connecting sugar moieties of the oligomer. The backbone structure or oligomer linkages of the ASOs described herein may include (but are not limited to) phosphorothioate, phosphorodithioate, phosphoroselenoate, phosphorodiselenoate, phosphoroanilothioate, phosphoraniladate, phosphoramidate, and the like. See e.g., LaPlanche et al., Nucleic Acids Res. 14:9081 (1986); Stec et al., J. Am. Chem. Soc. 106:6077 (1984), Stein et al., Nucleic Acids Res. 16:3209 (1988), Zon et al., Anti Cancer Drug Design 6:539 (1991); Zon et al., Oligonucleotides and Analogues: A Practical Approach, pp. 87-108 (F. Eckstein, Ed., Oxford University Press, Oxford England (1991)); Stec et al., U.S. Pat. No. 5,151,510; Uhlmann and Peyman, Chemical Reviews 90:543 (1990). In some embodiments, the backbone structure of the ASO does not contain phosphorous but rather contains peptide bonds, for example in a peptide nucleic acid (PNA), or linking groups including carbamate, amides, and linear and cyclic hydrocarbon groups. In some embodiments, the backbone modification is a phosphothioate linkage. In some embodiments, the backbone modification is a phosphoramidate linkage.

In some embodiments, the stereochemistry at each of the phosphorus internucleotide linkages of the ASO backbone is random. In some embodiments, the stereochemistry at each of the phosphorus internucleotide linkages of the ASO backbone is controlled and is not random. For example, U.S. Pat. App. Pub. No. 2014/0194610, “Methods for the Synthesis of Functionalized Nucleic Acids,” incorporated herein by reference, describes methods for independently selecting the handedness of chirality at each phosphorous atom in a nucleic acid oligomer. In some embodiments, an ASO used in the methods of the invention, including, but not limited to, any of the ASOs set forth herein in Table 1 or 3, comprises an ASO having phosphorus internucleotide linkages that are not random. In some embodiments, a composition used in the methods of the invention comprises a pure diastereomeric ASO. In some embodiments, a composition used in the methods of the invention comprises an ASO that has diastereomeric purity of at least about 90%, at least about 91%, at least about 92%, at least about 93%, at least about 94%, at least about 95%, at least about 96%, at least about 97%, at least about 98%, at least about 99%, about 100%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

In some embodiments, the ASO has a nonrandom mixture of Rp and Sp configurations at its phosphorus internucleotide linkages. For example, it has been suggested that a mix of Rp and Sp is required in antisense oligonucleotides to achieve a balance between good activity and nuclease stability (Wan, et al., 2014, “Synthesis, biophysical properties and biological activity of second-generation antisense oligonucleotides containing chiral phosphorothioate linkages,” Nucleic Acids Research 42(22): 13456-13468, incorporated herein by reference). In some embodiments, an ASO used in the methods of the invention, including, but not limited to, any of the ASOs set forth herein in Table 1 or 3, comprises about 5-100% Rp, at least about 5% Rp, at least about 10% Rp, at least about 15% Rp, at least about 20% Rp, at least about 25% Rp, at least about 30% Rp, at least about 35% Rp, at least about 40% Rp, at least about 45% Rp, at least about 50% Rp, at least about 55% Rp, at least about 60% Rp, at least about 65% Rp, at least about 70% Rp, at least about 75% Rp, at least about 80% Rp, at least about 85% Rp, at least about 90% Rp, or at least about 95% Rp, with the remainder Sp, or about 100% Rp. In some embodiments, an ASO used in the methods of the invention, including, but not limited to, any of the ASOs set forth herein in Table 1 or 3, comprises about 10% to about 100% Rp, about 15% to about 100% Rp, about 20% to about 100% Rp, about 25% to about 100% Rp, about 30% to about 100% Rp, about 35% to about 100% Rp, about 40% to about 100% Rp, about 45% to about 100% Rp, about 50% to about 100% Rp, about 55% to about 100% Rp, about 60% to about 100% Rp, about 65% to about 100% Rp, about 70% to about 100% Rp, about 75% to about 100% Rp, about 80% to about 100% Rp, about 85% to about 100% Rp, about 90% to about 100% Rp, or about 95% to about 100% Rp, about 20% to about 80% Rp, about 25% to about 75% Rp, about 30% to about 70% Rp, about 40% to about 60% Rp, or about 45% to about 55% Rp, with the remainder Sp.

In some embodiments, an ASO used in the methods of the invention, including, but not limited to, any of the ASOs set forth herein in Table 1 or 3, comprises about 5-100% Sp, at least about 5% Sp, at least about 10% Sp, at least about 15% Sp, at least about 20% Sp, at least about 25% Sp, at least about 30% Sp, at least about 35% Sp, at least about 40% Sp, at least about 45% Sp, at least about 50% Sp, at least about 55% Sp, at least about 60% Sp, at least about 65% Sp, at least about 70% Sp, at least about 75% Sp, at least about 80% Sp, at least about 85% Sp, at least about 90% Sp, or at least about 95% Sp, with the remainder Rp, or about 100% Sp. In some embodiments, an ASO used in the methods of the invention, including, but not limited to, any of the ASOs set forth herein in Table 1 or 3, comprises about 10% to about 100% Sp, about 15% to about 100% Sp, about 20% to about 100% Sp, about 25% to about 100% Sp, about 30% to about 100% Sp, about 35% to about 100% Sp, about 40% to about 100% Sp, about 45% to about 100% Sp, about 50% to about 100% Sp, about 55% to about 100% Sp, about 60% to about 100% Sp, about 65% to about 100% Sp, about 70% to about 100% Sp, about 75% to about 100% Sp, about 80% to about 100% Sp, about 85% to about 100% Sp, about 90% to about 100% Sp, or about 95% to about 100% Sp, about 20% to about 80% Sp, about 25% to about 75% Sp, about 30% to about 70% Sp, about 40% to about 60% Sp, or about 45% to about 55% Sp, with the remainder Rp.

Any of the ASOs described herein may contain a sugar moiety that comprises ribose or deoxyribose, as present in naturally occurring nucleotides, or a modified sugar moiety or sugar analog, including a morpholine ring. Non-limiting examples of modified sugar moieties include 2′ substitutions such as 2′-O-methyl (2′-O-Me), 2′-O-methoxyethyl (2′MOE), 2′-O-aminoethyl, 2′-Fluoro (2′F); N3′->P5′ phosphoramidate, 2′dimethylaminooxyethoxy, 2′dimethylaminoethoxyethoxy, 2′-guanidinidium, 2′-O-guanidinium ethyl, carbamate modified sugars, and bicyclic modified sugars. In some embodiments, the sugar moiety modification is selected from 2′-O-Me, 2′F, and 2′MOE. In some embodiments, the sugar moiety modification is an extra bridge bond, such as in a locked nucleic acid (LNA). In some embodiments the sugar analog contains a morpholine ring, such as phosphorodiamidate morpholino (PMO). In some embodiments, the sugar moiety comprises a ribofuransyl or 2′deoxyribofuransyl modification. In some embodiments, the sugar moiety comprises 2′4′-constrained 2′O-methyloxyethyl (cMOE) modifications. In some embodiments, the sugar moiety comprises cEt 2′, 4′ constrained 2′-0 ethyl BNA modifications. In some embodiments, the sugar moiety comprises tricycloDNA (tcDNA) modifications. In some embodiments, the sugar moiety comprises ethylene nucleic acid (ENA) modifications. In some embodiments, the sugar moiety comprises MCE modifications. Modifications are known in the art and described in the literature, e.g., by Jarver, et al., 2014, “A Chemical View of Oligonucleotides for Exon Skipping and Related Drug Applications,” Nucleic Acid Therapeutics 24(1): 37-47, incorporated by reference for this purpose herein.

In some examples, each monomer of the ASO is modified in the same way, for example each linkage of the backbone of the ASO comprises a phosphorothioate linkage or each ribose sugar moiety comprises a 2′O-methyl modification. Such modifications that are present on each of the monomer components of an ASO are referred to as “uniform modifications.” In some examples, a combination of different modifications may be desired, for example, an ASO may comprise a combination of phosphorodiamidate linkages and sugar moieties comprising morpholine rings (morpholinos). Combinations of different modifications to an ASO are referred to as “mixed modifications” or “mixed chemistries.”

In some embodiments, the ASO comprises one or more backbone modification. In some embodiments, the ASO comprises one or more sugar moiety modification. In some embodiments, the ASO comprises one or more backbone modification and one or more sugar moiety modification. In some embodiments, the ASO comprises 2′MOE modifications and a phosphorothioate backbone. In some embodiments, the ASO comprises a phosphorodiamidate morpholino (PMO). In some embodiments, the ASO comprises a peptide nucleic acid (PNA). Any of the ASOs or any component of an ASO (e.g., a nucleobase, sugar moiety, backbone) described herein may be modified in order to achieve desired properties or activities of the ASO or reduce undesired properties or activities of the ASO. For example, an ASO or one or more component of any ASO may be modified to enhance binding affinity to a target sequence on a pre-mRNA transcript; reduce binding to any non-target sequence; reduce degradation by cellular nucleases (i.e., RNase H); improve uptake of the ASO into a cell and/or into the nucleus of a cell; alter the pharmacokinetics or pharmacodynamics of the ASO; and modulate the half-life of the ASO.

In some embodiments, the ASOs are comprised of 2′-O-(2-methoxyethyl) (MOE) phosphorothioate-modified nucleotides. ASOs comprised of such nucleotides are especially well-suited to the methods disclosed herein; oligomers having such modifications have been shown to have significantly enhanced resistance to nuclease degradation and increased bioavailability, making them suitable, for example, for oral delivery in some embodiments described herein. See e.g., Geary et al., J Pharmacol Exp Ther. 2001; 296(3):890-7; Geary et al., J Pharmacol Exp Ther. 2001; 296(3):898-904.

Methods of synthesizing ASOs will be known to one of skill in the art. Alternatively or in addition, ASOs may be obtained from a commercial source.

Unless specified otherwise, the left-hand end of single-stranded nucleic acid (e.g., pre-mRNA transcript, oligonucleotide, ASO, etc.) sequences is the 5′ end and the left-hand direction of single or double-stranded nucleic acid sequences is referred to as the 5′ direction. Similarly, the right-hand end or direction of a nucleic acid sequence (single or double stranded) is the 3′ end or direction. Generally, a region or sequence that is 5′ to a reference point in a nucleic acid is referred to as “upstream,” and a region or sequence that is 3′ to a reference point in a nucleic acid is referred to as “downstream.” Generally, the 5′ direction or end of an mRNA is where the initiation or start codon is located, while the 3′ end or direction is where the termination codon is located. In some aspects, nucleotides that are upstream of a reference point in a nucleic acid may be designated by a negative number, while nucleotides that are downstream of a reference point may be designated by a positive number. For example, a reference point (e.g., an exon-exon junction in mRNA) may be designated as the “zero” site, and a nucleotide that is directly adjacent and upstream of the reference point is designated “minus one,” e.g., “−1,” while a nucleotide that is directly adjacent and downstream of the reference point is designated “plus one,” e.g., “+1.”

In other embodiments, the ASOs are complementary to (and bind to) a targeted region of a target AIC pre-mRNA that is downstream (in the 3′ direction) of the 5′ splice site of the alternative-intron in a target AIC pre-mRNA (e.g., the direction designated by positive numbers relative to the 5′ alternative splice site of the alternative-intron). In some embodiments, the ASOs are complementary to a targeted region of the target AIC pre-mRNA that is within the region +1 to +100 relative to the 5′ splice site of the alternative-intron. In some embodiments, the ASOs may be complementary to a targeted region of a target AIC pre-mRNA that is within the region between nucleotides +1 and +50 relative to the 5′ splice site of the alternative-intron. In some embodiments, the ASOs are complementary to a targeted region that is within the region +1 to +90, +1 to +80, 16 to +70, +1 to +60, +1 to +50, +1 to +40, +1 to +30, or +1 to +20 relative to 5′ splice site of the alternative-intron. In some embodiments, the target AIC pre-mRNA is encoded by a gene selected from: ARHGAP23, BRD1, DCHS1, EPB41L2, GPX8, HIVEP3, INVS, KIAA0319, NAIP, CD274, PTCH2, PTPRZ1, SON, ZCCHC2.

In other embodiments, the ASOs are complementary to (and bind to) a targeted region of a CD274 AIC pre-mRNA that is downstream (in the 3′ direction) of the 5′ splice site of the alternative-intron in a CD274 AIC pre-mRNA (e.g., the direction designated by positive numbers relative to the 5′ alternative splice site of the alternative-intron). In some embodiments, the ASOs are complementary to a targeted region of the CD274 AIC pre-mRNA that is within the region +1 to +100 relative to the 5′ splice site of the alternative-intron. In some embodiments, the ASOs may be complementary to a targeted region of a CD274 AIC pre-mRNA that is within the region between nucleotides +1 and +50 relative to the 5′ splice site of the alternative-intron. In some embodiments, the ASOs are complementary to a targeted region that is within the region +1 to +90, +1 to +80, 16 to +70, +1 to +60, +1 to +50, +1 to +40, +1 to +30, or +1 to +20 relative to 5′ splice site of the alternative-intron.

In some embodiments, the ASOs are complementary to a targeted region of a target AIC pre-mRNA that is upstream (in the 5′ direction) of the 3′ splice site of the alternative-intron in a target AIC pre-mRNA (e.g., in the direction designated by negative numbers) In some embodiments, the ASOs are complementary to a targeted region of the target AIC pre-mRNA that is within the region −1 to −100 relative to the 3′ splice site of the alternative-intron. In some embodiments, the ASOs are complementary to a targeted region of the target AIC pre-mRNA that is within the region −1 to −50 relative to the 3′ splice site of the alternative-intron. In some embodiments, the ASOs are complementary to a targeted region that is within the region −1 to −90, −1 to −80, −1 to −70, −1 to −60, −1 to −50, −1 to −40, or −1 to −30 relative to 3′ splice site of the alternative-intron. In some embodiments, the target AIC pre-mRNA is encoded by a gene selected from: ARHGAP23, BRD1, DCHS1, EPB41L2, GPX8, HIVEP3, INVS, KIAA0319, NAIP, CD274, PTCH2, PTPRZ1, SON, ZCCHC2.

In some embodiments, the ASOs are complementary to a targeted region of a CD274 AIC pre-mRNA that is upstream (in the 5′ direction) of the 3′ splice site of the alternative-intron in a CD274 AIC pre-mRNA (e.g., in the direction designated by negative numbers) In some embodiments, the ASOs are complementary to a targeted region of the CD274 AIC pre-mRNA that is within the region −1 to −100 relative to the 3′ splice site of the alternative-intron. In some embodiments, the ASOs are complementary to a targeted region of the CD274 AIC pre-mRNA that is within the region −1 to −50 relative to the 3′ splice site of the alternative-intron. In some embodiments, the ASOs are complementary to a targeted region that is within the region −1 to −90, −1 to −80, −1 to −70, −1 to −60, −1 to −50, −1 to −40, or −1 to −30 relative to 3′ splice site of the alternative-intron.

In some embodiments, the targeted region of the CD274 AIC pre-mRNA is within the region +100 relative to the 5′ splice site of the alternative-intron to −100 relative to the 3′ splice site of the alternative-intron.

In some embodiments, the ASOs are complementary to a targeted region of a target AIC pre-mRNA that is within the exon flanking the 5′ splice site (upstream) of the alternative-intron (e.g., the first portion of the exon within which the alternative-intron is located). In some embodiments, the ASOs are complementary to a targeted region of the target AIC pre-mRNA that is within the region +2e to −1e in the first portion of the exon flanking the 5′ splice site of the alternative-intron. In some embodiments, the ASOs are complementary to a targeted region of the target AIC pre-mRNA that is within the region −1e to −100e, −1e to −90e, −1e to −80e, −1e to −70e, −1e to −60e, −1e to −50e, −1 to −40e, −1e to −30e, or −1e to −20e relative to the 5′ splice site of the alternative-intron. In some embodiments, the target AIC pre-mRNA is encoded by a gene selected from: ARHGAP23, BRD1, DCHS1, EPB41L2, GPX8, HIVEP3, INVS, KIAA0319, NAIP, CD274, PTCH2, PTPRZ1, SON, ZCCHC2.

In some embodiments, the ASOs are complementary to a targeted region of a CD274 AIC pre-mRNA that is within the exon flanking the 5′ splice site (upstream) of the alternative-intron (e.g., the first portion of the exon within which the alternative-intron is located). In some embodiments, the ASOs are complementary to a targeted region of the CD274 AIC pre-mRNA that is within the region +2e to −1e in the first portion of the exon flanking the 5′ splice site of the alternative-intron. In some embodiments, the ASOs are complementary to a targeted region of the CD274 AIC pre-mRNA that is within the region −1e to −100e, −1e to −90e, −1e to −80e, −1e to −70e, −1e to −60e, −1e to −50e, −1 to −40e, −1e to −30e, or −1e to −20e relative to the 5′ splice site of the alternative-intron.

In some embodiments, the ASOs are complementary to a targeted region of a target AIC pre-mRNA that is within the exon flanking the 3′ splice site (downstream) of the alternative-intron (e.g., the second portion of the exon within which the alternative-intron is located). In some embodiments, the ASOs are complementary to a targeted region to the target AIC pre-mRNA that is within the region +1e to −4e in the exon flanking the 3′ splice site of the alternative-intron. In some embodiments, the ASOs are complementary to a targeted region of the target AIC pre-mRNA that is within the region +1e to +100e, +1e to +90e, +1e to +80e, +1e to +70e, +1e to +60e, +1e to +50e, +1e to +40e, +1e to +30e, or +1 to +20e relative to the 3′ splice site of the alternative-intron. In some embodiments, the target AIC pre-mRNA is encoded by a gene selected from: ARHGAP23, BRD1, DCHS1, EPB41L2, GPX8, HIVEP3, INVS, KIAA0319, NAIP, CD274, PTCH2, PTPRZ1, SON, ZCCHC2.

In some embodiments, the ASOs are complementary to a targeted region of a CD274 AIC pre-mRNA that is within the exon flanking the 3′ splice site (downstream) of the alternative-intron (e.g., the second portion of the exon within which the alternative-intron is located). In some embodiments, the ASOs are complementary to a targeted region to the CD274 AIC pre-mRNA that is within the region +1e to −4e in the exon flanking the 3′ splice site of the alternative-intron. In some embodiments, the ASOs are complementary to a targeted region of the CD274 AIC pre-mRNA that is within the region +1e to +100e, +1e to +90e, +1e to +80e, +1e to +70e, +1e to +60e, +1e to +50e, +1e to +40e, +1e to +30e, or +1e to +20e relative to the 3′ splice site of the alternative-intron.

In some embodiments, the therapeutic agent or ASO binds to a targeted region of CD274 AIC pre-mRNA. In some embodiments, the targeted region is within a sequence selected from SEQ ID NOs: 68, 69, and 71-76. In some embodiments, the targeted region of the AIC pre-mRNA to which the therapeutic agent or the ASO binds is located within exon 4 of CD274. In some embodiments, the ASO has a sequence selected from SEQ ID NOs: 1-67.

In some embodiments, the therapeutic agent or ASO binds to a targeted region of ARHGAP23 AIC pre-mRNA. In some embodiments, the targeted region is within a sequence selected from SEQ ID NOs: 77 and 91. In some embodiments, the targeted region of the AIC pre-mRNA to which the therapeutic agent or the ASO binds is located within an exon of ARHGAP23. In some embodiments, the ASO has a sequence selected from SEQ ID NOs: 105-153.

In some embodiments, the therapeutic agent or ASO binds to a targeted region of BRD1 AIC pre-mRNA. In some embodiments, the targeted region is within a sequence selected from SEQ ID NOs: 78 and 92. In some embodiments, the targeted region of the AIC pre-mRNA to which the therapeutic agent or the ASO binds is located within an exon of BRD1. In some embodiments, the ASO has a sequence selected from SEQ ID NOs: 154-217.

In some embodiments, the therapeutic agent or ASO binds to a targeted region of DCHS1 AIC pre-mRNA. In some embodiments, the targeted region is within a sequence selected from SEQ ID NOs: 79 and 93. In some embodiments, the targeted region of the AIC pre-mRNA to which the therapeutic agent or the ASO binds is located within an exon of DCHS1. In some embodiments, the ASO has a sequence selected from SEQ ID NOs: 218-334.

In some embodiments, the therapeutic agent or ASO binds to a targeted region of EPB41L2 AIC pre-mRNA. In some embodiments, the targeted region is within a sequence selected from SEQ ID NOs: 80 and 94. In some embodiments, the targeted region of the AIC pre-mRNA to which the therapeutic agent or the ASO binds is located within an exon of EPB41L2. In some embodiments, the ASO has a sequence selected from SEQ ID NOs: 335-406.

In some embodiments, the therapeutic agent or ASO binds to a targeted region of GPX8 AIC pre-mRNA. In some embodiments, the targeted region is within a sequence selected from SEQ ID NOs: 81 and 95. In some embodiments, the targeted region of the AIC pre-mRNA to which the therapeutic agent or the ASO binds is located within an exon of GPX8. In some embodiments, the ASO has a sequence selected from SEQ ID NOs: 407-443.

In some embodiments, the therapeutic agent or ASO binds to a targeted region of HIVEP3 AIC pre-mRNA. In some embodiments, the targeted region is within a sequence selected from SEQ ID NOs: 82 and 96. In some embodiments, the targeted region of the AIC pre-mRNA to which the therapeutic agent or the ASO binds is located within an exon of HIVEP3. In some embodiments, the ASO has a sequence selected from SEQ ID NOs: 444-1027.

In some embodiments, the therapeutic agent or ASO binds to a targeted region of INVS AIC pre-mRNA. In some embodiments, the targeted region is within a sequence selected from SEQ ID NOs: 83-97. In some embodiments, the targeted region of the AIC pre-mRNA to which the therapeutic agent or the ASO binds is located within an exon of INVS. In some embodiments, the ASO has a sequence selected from SEQ ID NOs: 1028-1112.

In some embodiments, the therapeutic agent or ASO binds to a targeted region of KA10319 AIC pre-mRNA. In some embodiments, the targeted region is within a sequence selected from SEQ ID NOs: 84-98. In some embodiments, the targeted region of the AIC pre-mRNA to which the therapeutic agent or the ASO binds is located within an exon of KA10319. In some embodiments, the ASO has a sequence selected from SEQ ID NOs: 1113-1212.

In some embodiments, the therapeutic agent or ASO binds to a targeted region of NAIP AIC pre-mRNA. In some embodiments, the targeted region is within a sequence selected from SEQ ID NOs: 85-99. In some embodiments, the targeted region of the AIC pre-mRNA to which the therapeutic agent or the ASO binds is located within an exon of NAIP. In some embodiments, the ASO has a sequence selected from SEQ ID NOs: 1213-1501.

In some embodiments, the therapeutic agent or ASO binds to a targeted region of PTCH2 AIC pre-mRNA. In some embodiments, the targeted region is within a sequence selected from SEQ ID NOs: 86-100. In some embodiments, the targeted region of the AIC pre-mRNA to which the therapeutic agent or the ASO binds is located within an exon of PTCH2. In some embodiments, the ASO has a sequence selected from SEQ ID NOs: 1502-1547.

In some embodiments, the therapeutic agent or ASO binds to a targeted region of PTPRZ1 AIC pre-mRNA. In some embodiments, the targeted region is within a sequence selected from SEQ ID NOs: 87-101. In some embodiments, the targeted region of the AIC pre-mRNA to which the therapeutic agent or the ASO binds is located within an exon of PTPRZ1. In some embodiments, the ASO has a sequence selected from SEQ ID NOs: 1548-2039.

In some embodiments, the therapeutic agent or ASO binds to a targeted region of SON AIC pre-mRNA. In some embodiments, the targeted region is within a sequence selected from SEQ ID NOs: 88, 89, 102 and 103. In some embodiments, the targeted region of the AIC pre-mRNA to which the therapeutic agent or the ASO binds is located within an exon of SON. In some embodiments, the ASO has a sequence selected from SEQ ID NOs: 2040-3458.

In some embodiments, the therapeutic agent or ASO binds to a targeted region of ZCCHC2 AIC pre-mRNA. In some embodiments, the targeted region is within a sequence selected from SEQ ID NOs: 90 and 104. In some embodiments, the targeted region of the AIC pre-mRNA to which the therapeutic agent or the ASO binds is located within an exon of ZCCHC2. In some embodiments, the ASO has a sequence selected from SEQ ID NOs: 3459-3651.

The ASOs may be of any length suitable for specific binding and effective modulation (e.g., inhibition or enhancement) of splicing. In some embodiments, the ASOs consist of 8 to 50 nucleobases. For example, the ASO may be 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 nucleobases in length. In some embodiments, the ASOs consist of more than 50 nucleobases. In some embodiments, the ASO is from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 13 to 50 nucleobases, 13 to 40 nucleobases, 13 to 35 nucleobases, 13 to 30 nucleobases, 13 to 25 nucleobases, 13 to 20 nucleobases, 14 to 50 nucleobases, 14 to 40 nucleobases, 14 to 35 nucleobases, 14 to 30 nucleobases, 14 to 25 nucleobases, 14 to 20 nucleobases, 15 to 50 nucleobases, 15 to 40 nucleobases, 15 to 35 nucleobases, 15 to 30 nucleobases, 15 to 25 nucleobases, 15 to 20 nucleobases, 20 to 50 nucleobases, 20 to 40 nucleobases, 20 to 35 nucleobases, 20 to 30 nucleobases, 20 to 25 nucleobases, 25 to 50 nucleobases, 25 to 40 nucleobases, 25 to 35 nucleobases, or 25 to 30 nucleobases in length. In some embodiments, the ASOs are 18 nucleotides in length. In some embodiments, the ASOs are 15 nucleotides in length. In some embodiments, the ASOs are 25 nucleotides in length.

In some embodiments, two or more ASOs with different chemistries but complementary to the same targeted region of the AIC pre-mRNA are used. In some embodiments, two or more ASOs that are complementary to different targeted regions of the AIC pre-mRNA are used.

In some embodiments, the ASOs of the invention are chemically linked to one or more moieties or conjugates, e.g., a targeting moiety or other conjugate that enhances the activity or cellular uptake of the oligonucleotide. Such moieties include, but are not limited to, a lipid moiety, e.g., as a cholesterol moiety, a cholesteryl moiety, an aliphatic chain, e.g., dodecandiol or undecyl residues, a polyamine or a polyethylene glycol chain, or adamantane acetic acid. Oligonucleotides comprising lipophilic moieties and preparation methods have been described in the published literature. In some embodiments, the antisense oligonucleotide is conjugated with a moiety including, but not limited to, an abasic nucleotide, a polyether, a polyamine, a polyamide, a peptides, a carbohydrate, e.g., N-acetylgalactosamine (GalNAc), N—Ac-Glucosamine (GluNAc), or mannose (e.g., mannose-6-phosphate), a lipid, or a polyhydrocarbon compound. Conjugates can be linked to one or more of any nucleotides comprising the antisense oligonucleotide at any of several positions on the sugar, base or phosphate group, as understood in the art and described in the literature, e.g., using a linker. Linkers can include a bivalent or trivalent branched linker. In some embodiments, the conjugate is attached to the 3′ end of the antisense oligonucleotide. Methods of preparing oligonucleotide conjugates are described, e.g., in U.S. Pat. No. 8,450,467, “Carbohydrate conjugates as delivery agents for oligonucleotides,” incorporated by reference herein.

In some embodiments, the nucleic acid to be targeted by an ASO is a CD274 AIC pre-mRNA expressed in a cell, such as a eukaryotic cell. In some embodiments, the term “cell” may refer to a population of cells. In some embodiments, the cell is in a subject. In some embodiments, the cell is isolated from a subject. In some embodiments, the cell is ex vivo. In some embodiments, the cell is in a tissue ex vivo. In some embodiments, the cell is in an organ ex vivo. In some embodiments, the cell is a condition or disease-relevant cell or a cell line. In some embodiments, the cell is in vitro (e.g., in cell culture).

In some embodiments, the therapeutic agent or the ASO inhibits splicing of the alternative-intron from the AIC pre-mRNA encoding the target protein. In some embodiments, the therapeutic agent or the ASO increases the level of processed mRNA encoding the target protein in the cell. In some embodiments, the level of processed mRNA encoding the target protein in the cell contacted with the therapeutic agent or the ASO is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell, e.g., a cell not contacted with a therapeutic agent or an ASO or a cell contacted with a therapeutic agent or an ASO that is not complementary to the targeted region of the AIC pre-mRNA.

In some embodiments, the therapeutic agent or ASO increases the expression of the target protein in the cell. In some embodiments, the level of the target protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of the target protein in a control cell, e.g., a cell not contacted with a therapeutic agent or an ASO or a cell contacted with a therapeutic agent or an ASO that is not complementary to the targeted region of the AIC pre-mRNA.

In some embodiments, the therapeutic agent or the ASO promotes or enhances splicing of the alternative-intron from the AIC pre-mRNA encoding the target protein. In some embodiments, the therapeutic agent or the ASO decreases the level of processed mRNA encoding the target protein in the cell. In some embodiments, the level of processed mRNA encoding the target protein in the cell contacted with the therapeutic agent is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell, e.g., a cell not contacted with a therapeutic agent or an ASO or a cell contacted with a therapeutic agent or an ASO that is not complementary to the targeted region of the AIC pre-mRNA.

In some embodiments, the therapeutic agent or ASO decreases the expression of the target protein in the cell. In some embodiments, the level of the target protein in the cell contacted with the therapeutic agent is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of the target protein in a control cell, e.g., a cell not contacted with a therapeutic agent or an ASO or a cell contacted with a therapeutic agent or an ASO that is not complementary to the targeted region of the AIC pre-mRNA.

### Diseases and Disorders

In some embodiments, the methods and compositions of the present invention are used to treat any immune disease or immune disorder in a subject in need thereof. In some embodiments, an immune disease or an immune disorder treated using the methods and compositions of the present invention is, e.g., an autoimmune disease or an autoimmune disorder, an inflammatory disease or an inflammatory disorder, a chronic infection, graft-versus-host disease (GVHD), a transplant rejection, or a T cell proliferative disorder. In some embodiments, the immune disease or the immune disorder is an autoimmune disease or an autoimmune disorder or an inflammatory disease or an inflammatory disorder selected from: multiple sclerosis, inflammatory bowel disease, autoimmune hepatitis, kidney inflammation, rheumatoid arthritis, psoriasis, lupus nephritis, corneal transplant, and uveitis. In some embodiments, the immune disease of the immune disorder is a disorder that is mediated by T cells, B cells, or NK cells.

An autoimmune disease or an autoimmune disorder is a condition characterized by cellular, tissue, and/or organ injury caused by an immunologic reaction of a subject to its own cells, tissues, and/or organs. An inflammatory disease or an inflammatory disorder refers to a condition in a subject characterized by inflammation, including, e.g., chronic inflammation. Autoimmune disorders may or may not be associated with inflammation. Inflammation may or may not be caused by an autoimmune disorder. Therefore, certain disorders may be characterized as both autoimmune and inflammatory disorders. An autoimmune disease or an autoimmune disorder treated using the methods and compositions of the invention may be, e.g., alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, diabetes, eosinophilic fascites, fibromyalgia-fibromyositis, glomerulonephritis, Graves' disease, Guillain-Barre, Hashimoto's thyroiditis, Henoch-Schonlein purpura, idiopathic pulmonary fibrosis, idiopathic/autoimmune thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, pemphigus-related disorders (e.g., pemphigus vulgaris), pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjogren's syndrome, stiff-man syndrome, systemic lupus erythematosis (SLE), Sweet's syndrome, Still's disease, lupus erythematosus, takayasu arteritis, temporal arteristis/giant cell arteritis, ulcerative colitis, uveitis, vasculitides such as dermatitis herpetiformis vasculitis, vitiligo, and Wegener's granulomatosis.

In some embodiments, an immune disease or an immune disorder treated using the methods and compositions of the present invention is an inflammatory disease or an inflammatory disorder, for example, inflammation accompanying arthritis (e.g., rheumatoid arthritis, osteoarthritis), pneumonia, hepatitis (including viral hepatitis), inflammation accompanying infectious diseases, inflammatory bowel diseases, intestinal enteritis, nephritis (e.g., glomerular nephritis, nephrofibrosis), gastritis, angiitis, pancreatitis, peritonitis, bronchitis, myocarditis, cerebritis, inflammation in postischemic reperfusion injury (myocardial ischemic reperfusion injury), inflammation attributed to immune rejection after transplantation of tissue and organ, burn, various skin inflammation (psoriasis, allergic contact-type dermatitis, lichen planus), inflammation in multiple organ failure, inflammation after operation of PTCA or PTCR, inflammation accompanying arteriosclerosis, autoimmune thyroiditis, asthma, encephilitis, chronic obstructive pulmonary disease (COPD), allergic disorders, septic shock, pulmonary fibrosis, undifferentitated spondyloarthropathy, undifferentiated arthropathy, spondyloarthropathies (e.g., psoriatic arthritis, ankylosing spondylitis, and Reiter's Syndrome or reactive arthritis), inflammatory osteolysis, Wilson's disease and chronic inflammation resulting from chronic viral or bacterial infections.

In some embodiments, an immune disease or an immune disorder treated using the methods and compositions of the present invention is a chronic inflammatory disease or a chronic inflammatory disorder selected from, e.g., inflammatory bowel disease (including, e.g., Crohn's disease and ulcerative colitis), Grave's disease, Hashimoto's thyroiditis, allergic contact-type dermatitis, chronic inflammatory dermatosis (e.g., lichen planus), and diabetes mellitis.

In some embodiments, an immune disease or an immune disorder treated using the methods and compositions of the present invention is, e.g., toxic shock syndrome, inflammatory bowel disease, allosensitization due to blood transfusion, or a T-cell dependent B-cell-mediated disease, osteoarthritis (OA), graft versus host reaction (GVH reaction), graft versus host disease (GVHD), immune rejection accompanying transplantation of a tissue (e.g., skin, cornea, bone) or organ (e.g., liver, heart, lung, kidney, pancreas), immune response triggered by a foreign antigen or autoantigen (for example, production of antibodies against said antigen, cell proliferation, production of cytokines), or a disorder caused by abnormal intestinal immunity (e.g., inflammatory intestinal disorders, Crohn's disease, ulcerative colitis, and alimentary allergy).

Autoimmune diseases can affect any tissue or body part, including but not limited to the heart, brain, nerves, muscles, skin, eyes, joints, lungs, kidneys, glands (e.g., thyroid), the digestive tract, and blood vessels. The autoimmune disease systemic lupus erythematosus can affect the skin, joints, kidneys, heart, nerves, blood vessels, and other tissues. Type 1 diabetes can affect, e.g., glands, eyes, kidneys, and muscles. In some embodiments, a subject treated using the methods and compositions of the invention has a propensity to develop an autoimmune disease at the time of treatment. In some embodiments, a subject treated using the methods and compositions of the invention is treated preventively.

In some embodiments, a subject having an autoinflammatory disorder is treated using the methods and compositions of the invention. Autoinflammatory disorders are characterized by intense episodes of inflammation that result in such symptoms as fever, rash, or joint swelling. In some cases, an inflammatory disorder arises due to a chronic condition. For example, nonalcoholic steatohepatitis (NASH) is an inflammatory disorder related to fatty liver disease. Other inflammatory disorders contemplated for treatment using the methods and compositions of the present invention include, e.g., Familial Mediterranean Fever (FMF), Neonatal Onset Multisystem Inflammatory Disease (NOMID), Tumor Necrosis Factor (TNF) Receptor-Associated Periodic Syndrome (TRAPS), Deficiency of the Interleukin-1 Receptor Antagonist (DIRA), poststreptococcal and autiommune renal failure, septic shock, systemic inflammatory response syndrome (SIRS), adult respiratory distress syndrome (ARDS), and inflammation due to envenomation.

Examples of autoimmune disorders treated with the present invention include, e.g., systemic lupus erythematosus, thyroiditis, uveitis, vitiligo, granulomatosis with polyangiitis (Wegener's), multiple sclerosis, systemic lupus erythematosus/lupus nephritis, Hashimoto's thyroiditis, autoimmune hepatitis, myasthenia gravis, myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjogren's syndrome, alopecia areata, autoimmune hemolytic anemia, autoimmune hepatitis, dermatomyositis, diabetes (type 1), juvenile idiopathic arthritis, glomerulonephritis, Graves' disease, Guillain-Barré syndrome, idiopathic thrombocytopenic purpura, Behcet disease, systemic lupus erythematosus, multiple sclerosis (systemic scleroderma and progressive systemic scleroderma), scleroderma, polymyositis, dermatomyositis, periarteritis nodosa (polyarteritis nodosa and microscopic polyangiitis), aortitis syndrome (Takayasu arteritis), malignant rheumatoid arthritis, rheumatoid arthritis, mixed connective tissue disease, adult-onset Still's disease, allergic granulomatous angiitis, hypersensitivity angiitis, Cogan's syndrome, RS3PE, temporal arteritis, polymyalgia rheumatica, fibromyalgia syndrome, antiphospholipid antibody syndrome, eosinophilic fasciitis, IgG4-related diseases (e.g., primary sclerosing cholangitis and autoimmune pancreatitis), Guillain-Barre syndrome, myasthenia gravis, chronic atrophic gastritis, autoimmune hepatitis, primary biliary cirrhosis, aortitis syndrome, Goodpasture's syndrome, rapidly progressive glomerulonephritis, megaloblastic anemia, autoimmune hemolytic anemia, autoimmune neutropenia, idiopathic thrombocytopenic purpura, Graves' disease (hyperthyroidism), Hashimoto's thyroiditis, autoimmune adrenal insufficiency, primary hypothyroidism, idiopathic Addison's disease (chronic adrenal insufficiency), type I diabetes mellitus, chronic discoid lupus erythematosus, localized scleroderma, psoriasis, psoriatic arthritis, pemphigus, pemphigoid, herpes gestationis, linear IgA bullous skin disease, epidermolysis bullosa acquisita, alopecia areata, vitiligo, Harada disease, autoimmune optic neuropathy, idiopathic azoospermia, recurrent fetal loss, and inflammatory bowel diseases (ulcerative colitis and Crohn's disease).

In some embodiments, the methods of the present invention involve contacting cells of a subject with an ASO. In some embodiments, the contacted cell is an immune system cell at any state of differentiation. In some embodiments, the cell is a stem cell, a progenitor cell, a dendritic cell, a macrophage, a peritoneal B1 B cell, a memory B cell, a bone marrow (BM)-derived mast cell, a hematopoietic cell, a non-hemopoietic cell, a B cell, or a T cell. In some embodiments, the immune system cell is a T cell. In some embodiments, the T cell is a CD4+ T cell, a CD8+ T cell, or a killer CD8+ T cell. In some embodiments, the T cell is an activated T cell. In some embodiments, the T cell is a pathogenic CD4+T_{H}1 or T_{H}17 effector cell.

In some embodiments, a cell contacted in the methods of the present invention is a non-hematopoietic cell, e.g., a vascular endothelial cell, a fibroblastic reticular cell, an epithelial cell, a pancreatic islet cell, an astrocyte, a neuron, a Schwann cell of CNS/PNS, a hepatocyte, a cornea, a renal cell or a cell at a site of immune privilege including a trophoblast in the placenta or a retinal pigment epithelial cell or a neuron in the eye, or any other appropriate cell type known in the art and identified for targeting in the treatment of an immunological disorder. In some embodiments, the cell contacted is a cell type that expresses a target protein. In some embodiments, the cell contacted is a cell type that expresses PD-L1.

In some embodiments of the present invention, a cell is obtained from a subject in need thereof and modified ex vivo with ASOs to induce target protein expression (e.g., PD-L1) and adoptively transferred to a subject. In other embodiments, a cell, e.g., a T_{H}1 cell, can be obtained from a subject in need thereof and induced to convert to a T_{REG} cell and adoptively transferred to a said patient in need thereof. In some cases, a subject in need thereof has a transplant-related autoimmune disease. In some embodiments, GVHD is treated by inducing PD-L1 expression in a target cell population. In some embodiments, an immune disorder is treated by the methods of the invention using ex vivo modified cells. In some embodiments, an immune disorder is treated with a systemic infusion of a therapy of the present invention.

In some embodiments, the genetic disorder or condition can be an autosomal dominant disorder, an autosomal recessive disorder, X-linked dominant disorder, X-linked recessive disorder, Y-linked disorder, mitochondrial disease, or multifactorial or polygenic disorder. Sometimes, a hereditary disease can also be characterized as an autosomal dominant, autosomal recessive, X-linked dominant, X-linked recessive, Y-linked, mitochondrial, or multifactorial or polygenic hereditary disease. Autosomal dominant disorder, an autosomal recessive disorder, X-linked dominant disorder, X-linked recessive disorder, Y-linked disorder, mitochondrial disease, or multifactorial or polygenic disorder can be characterized by an impaired production of a protein. Autosomal dominant disorder, an autosomal recessive disorder, X-linked dominant disorder, X-linked recessive disorder, Y-linked disorder, mitochondrial disease, or multifactorial or polygenic disorder can be characterized by a defective splicing. A subject with an autosomal dominant disorder, an autosomal recessive disorder, X-linked dominant disorder, X-linked recessive disorder, Y-linked disorder, mitochondrial disease, or multifactorial or polygenic disorder can have a genome that can comprise a copy of a gene that comprises an exon that when properly transcribed into fully processed mRNA can encode the full-length functional for of the protein. A subject with an autosomal dominant disorder, an autosomal recessive disorder, X-linked dominant disorder, X-linked recessive disorder, Y-linked disorder, mitochondrial disease, or multifactorial or polygenic disorder can have a genome that comprises a copy of a gene that can comprise a copy of a gene that comprises a set of exons that when properly transcribed into fully processed mRNA can encode the full-length functional form of the protein. A subject with an autosomal dominant disorder, an autosomal recessive disorder, X-linked dominant disorder, X-linked recessive disorder, Y-linked disorder, mitochondrial disease, or multifactorial or polygenic disorder can have a genome that can comprise a defective copy of the gene, which can be incapable of producing a full-length functional form of the protein.

Exemplary hereditary disease can include achondroplasia, hereditary hemochromatosis, Down Syndrome, hereditary spherocytosis, Tay-Sachs Disease, Usher syndrome, hereditary fructose intolerance, hemophilia, muscular dystrophy (e.g., Duchenne muscular dystrophy or DMD), polygenic disorders, breast cancer, ovarian cancer, Parkinson's disease, Bardet-Biedl syndrome, Prader-Willi syndrome, diabetes, heart disease, arthritis, motor neuron disease, albinism, Cri-du-Chat syndrome, cystic fibrosis, fragile X syndrome, galactosemia, Huntington's disease, Jackson-Weiss syndrome, Klinefelter syndrome, Krabbe disease, Langer-Giedion syndrome, Lesch-Nyhan syndrome, Marfan syndrome, myotonic dystrophy, Nail-Patella syndrome, neurofibromatosis, Noonan syndrome, triple X syndrome, osteogenesis imperfecta, Patau syndrome, phenylketonuria, porphyria, retinoblastoma, Rett syndrome, sickle cell disease, Turner syndrome, Usher syndrome, Von Hippel-Lindau syndrome, Waardenburg syndrome, Wilson's disease, xeroderma pigmentosum, XXXX syndrome, or YY syndrome.

A hereditary disease such as for example: achondroplasia, hereditary hemochromatosis, Down Syndrome, hereditary spherocytosis, Tay-Sachs Disease, Usher syndrome, hereditary fructose intolerance, hemophilia, muscular dystrophy (e.g., Duchenne muscular dystrophy or DMD), polygenic disorders, breast cancer, ovarian cancer, Parkinson's disease, Bardet-Biedl syndrome, Prader-Willi syndrome, diabetes, heart disease, arthritis, motor neuron disease, albinism, Cri-du-Chat syndrome, cystic fibrosis, fragile X syndrome, galactosemia, Huntington's disease, Jackson-Weiss syndrome, Klinefelter syndrome, Krabbe disease, Langer-Giedion syndrome, Lesch-Nyhan syndrome, Marfan syndrome, myotonic dystrophy, Nail-Patella syndrome, neurofibromatosis, Noonan syndrome, triple X syndrome, osteogenesis imperfecta, Patau syndrome, phenylketonuria, porphyria, retinoblastoma, Rett syndrome, sickle cell disease, Turner syndrome, Usher syndrome, Von Hippel-Lindau syndrome, Waardenburg syndrome, Wilson's disease, xeroderma pigmentosum, XXXX syndrome, or YY syndrome can be characterized by an impaired production of a protein, or by a defective splicing. A hereditary disease such as for example: achondroplasia, hereditary hemochromatosis, Down Syndrome, hereditary spherocytosis, Tay-Sachs Disease, Usher syndrome, hereditary fructose intolerance, hemophilia, muscular dystrophy (e.g., Duchenne muscular dystrohy or DMD), polygenic disorders, breast cancer, ovarian cancer, Parkinson's disease, Bardet-Biedl syndrome, Prader-Willi syndrome, diabetes, heart disease, arthritis, motor neuron disease, albinism, Cri-du-Chat syndrome, cystic fibrosis, fragile X syndrome, galactosemia, Huntington's disease, Jackson-Weiss syndrome, Klinefelter syndrome, Krabbe disease, Langer-Giedion syndrome. Lesch-Nyhan syndrome, Marfan syndrome, myotonic dystrophy, Nail-Patella syndrome, neurofibromatosis, Noonan syndrome, triple X syndrome, osteogenesis imperfecta, Patau syndrome, phenylketonuria, porphyria, retinoblastoma, Rett syndrome, sickle cell disease, Turner syndrome, Usher syndrome, Von Hippel-Lindau syndrome, Waardenburg syndrome, Wilson's disease, xeroderma pigmentosum, XXXX syndrome, or YY syndrome can comprise a copy of a gene that comprises an exon that when properly transcribed into fully processed mRNA can encode the full-length functional for of the protein, can comprises a copy of a gene that can comprise a copy of a gene that comprises a set of exons that when properly transcribed into fully processed mRNA can encode the full-length functional form of the protein, or can comprise a defective copy of the gene, which can be incapable of producing a full-length functional form of the protein.

As described above, the genetic disorder or condition can be an autosomal dominant disorder, an autosomal recessive disorder, X-linked dominant disorder, X-linked recessive disorder, Y-linked disorder, mitochondrial disease, or multifactorial or polygenic disorder. The genetic disorder or condition can be an autosomal dominant disorder, an autosomal recessive disorder, X-linked dominant disorder, X-linked recessive disorder, Y-linked disorder, mitochondrial disease, or multifactorial or polygenic disorder can be a disorder characterized by an impaired production of a protein or by a defective splicing.

Exemplary autosomal dominant disorder can include Huntington's disease, neurofibromatosis type 1, neurofibromatosis type 2, Marfan syndrome, hereditary nonpolyposis colorectal cancer, hereditary multiple exostoses, Tuberous sclerosis, Von Willebrand disease, or acute intermittent porphyria.

### Pharmaceutical Compositions

Pharmaceutical compositions or formulations comprising the antisense oligomer (ASO) of the described compositions and for use in any of the described methods can be prepared according to conventional techniques well known in the pharmaceutical industry and described in the published literature. In some embodiments, a pharmaceutical composition or formulation for treating a subject comprises an effective amount of any ASO as described above, or a pharmaceutically acceptable salt, solvate, hydrate, or ester thereof, and a pharmaceutically acceptable diluent. The ASO of a pharmaceutical formulation may further comprise a pharmaceutically acceptable excipient, diluent, or carrier.

Pharmaceutically acceptable salts are suitable for use in contact with the tissues of humans and lower animals without undue toxicity, irritation, allergic response, etc., and are commensurate with a reasonable benefit/risk ratio. The salts can be prepared in situ during the final isolation and purification of the compounds, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

In some embodiments, the compositions are formulated into any of many possible dosage forms such as, but not limited to, tablets, capsules, gel capsules, liquid syrups, soft gels, suppositories, and enemas. In some embodiments, the compositions are formulated as suspensions in aqueous, non-aqueous, or mixed media. Aqueous suspensions may further contain substances that increase the viscosity of the suspension including, for example, sodium carboxymethylcellulose, sorbitol, and/or dextran. The suspension may also contain stabilizers. In some embodiments, a pharmaceutical formulation or composition of the present invention includes, but is not limited to, a solution, emulsion, microemulsion, foam, or liposome-containing formulation (e.g., cationic or noncationic liposomes).

The pharmaceutical composition or formulation of the present invention may comprise one or more penetration enhancer, carrier, excipient, or other active or inactive ingredients as appropriate and well known to those of skill in the art or described in the published literature. In some embodiments, liposomes also include sterically stabilized liposomes, e.g., liposomes comprising one or more specialized lipids. These specialized lipids result in liposomes with enhanced circulation lifetimes. In some embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations, and emulsions is well known in the art. In some embodiments, the present invention employs a penetration enhancer to effect the efficient delivery of the ASO, e.g., to aid diffusion across cell membranes and/or enhance the permeability of a lipophilic drug. In some embodiments, the penetration enhancer is a surfactant, fatty acid, bile salt, chelating agent, or non-chelating nonsurfactant.

In some embodiments, the pharmaceutical formulation comprises multiple ASOs. In some embodiments, the ASO is administered in combination with another drug or therapeutic agent. In some embodiments, the ASO is administered with one or more agents capable of promoting penetration of the subject ASO across the blood-brain barrier by any method known in the art. For example, delivery of agents by administration of an adenovirus vector to motor neurons in muscle tissue is described in U.S. Pat. No. 6,632,427, “Adenoviral-vector-mediated gene transfer into medullary motor neurons,” incorporated herein by reference. Delivery of vectors directly to the brain, e.g., the striatum, the thalamus, the hippocampus, or the substantia nigra, is described, e.g., in U.S. Pat. No. 6,756,523, “Adenovirus vectors for the transfer of foreign genes into cells of the central nervous system particularly in brain,” incorporated herein by reference.

In some embodiments, the ASOs are linked or conjugated with agents that provide desirable pharmaceutical or pharmacodynamic properties. In some embodiments, the ASO is coupled to a substance, known in the art to promote penetration or transport across the blood-brain barrier, e.g., an antibody to the transferrin receptor. In some embodiments, the ASO is linked with a viral vector, e.g., to render the antisense compound more effective or increase transport across the blood-brain barrier. In some embodiments, osmotic blood brain barrier disruption is assisted by infusion of sugars, e.g., meso erythritol, xylitol, D(+) galactose, D(+) lactose, D(+) xylose, dulcitol, myo-inositol, L(−) fructose, D(−) mannitol, D(+) glucose, D(+) arabinose, D(−) arabinose, cellobiose, D(+) maltose, D(+) raffinose, L(+) rhamnose, D(+) melibiose, D(−) ribose, adonitol, D(+) arabitol, L(−) arabitol, D(+) fucose, L(−) fucose, D(−) lyxose, L(+) lyxose, and L(−) lyxose, or amino acids, e.g., glutamine, lysine, arginine, asparagine, aspartic acid, cysteine, glutamic acid, glycine, histidine, leucine, methionine, phenylalanine, proline, serine, threonine, tyrosine, valine, and taurine. Methods and materials for enhancing blood brain barrier penetration are described, e.g., in U.S. Pat. No. 4,866,042, “Method for the delivery of genetic material across the blood brain barrier,” U.S. Pat. No. 6,294,520, “Material for passage through the blood-brain barrier,” and U.S. Pat. No. 6,936,589, “Parenteral delivery systems,” each incorporated herein by reference.

In some embodiments, the ASOs of the invention are chemically linked to one or more moieties or conjugates, e.g., a targeting moiety or other conjugate that enhances the activity or cellular uptake of the oligonucleotide. Such moieties include, but are not limited to, a lipid moiety, e.g., as a cholesterol moiety, a cholesteryl moiety, an aliphatic chain, e.g., dodecandiol or undecyl residues, a polyamine or a polyethylene glycol chain, or adamantane acetic acid. Oligonucleotides comprising lipophilic moieties and preparation methods have been described in the published literature. In some embodiments, the ASO is conjugated with a moiety including, but not limited to, an abasic nucleotide, a polyether, a polyamine, a polyamide, a peptide, a carbohydrate, e.g., N-acetylgalactosamine (GalNAc), N—Ac-Glucosamine (GluNAc), or mannose (e.g., mannose-6-phosphate), a lipid, or a polyhydrocarbon compound. Conjugates can be linked to one or more of any nucleotides comprising the ASO at any of several positions on the sugar, base or phosphate group, as understood in the art and described in the literature, e.g., using a linker. Linkers can include a bivalent or trivalent branched linker. In some embodiments, the conjugate is attached to the 3′ end of the ASO. Methods of preparing oligonucleotide conjugates are described, e.g., in U.S. Pat. No. 8,450,467, “Carbohydrate conjugates as delivery agents for oligonucleotides,” incorporated by reference herein.

### Treatment of Subjects

Any of the compositions provided herein may be administered to an individual. An “individual” may be used interchangeably with a “subject” or a “patient.” An individual may be a mammal, for example, a human or animal such as a non-human primate, a rodent, a rabbit, a rat, a mouse, a horse, a donkey, a goat, a cat, a dog, a cow, a pig, or a sheep. In some embodiments, the individual is a human. In some embodiments, the individual is a fetus, an embryo, or a child. In some embodiments, the individual is a non-human animal. In other embodiments, the individual may be another eukaryotic organism, such as a plant. In some embodiments, the compositions provided herein are administered to a cell ex vivo. In some embodiments, the compositions provided herein are administered to an organ or a tissue ex vivo (e.g., by perfusion or other means) prior to transplantation of the organ or the tissue.

In some embodiments, the compositions provided herein are administered to an individual as a method of treating a disease or a disorder. In some embodiments, the individual has an autoimmune or inflammatory disease, such as any of the diseases described herein. In some embodiments, the individual is at risk of having a disease, such as any of the diseases described herein. In some embodiments, the individual is at an increased risk of having a disease or a disorder caused by insufficient amount of a protein or insufficient activity of a protein. If an individual is “at an increased risk” of having a disease or a disorder caused insufficient amount of a protein or insufficient activity of a protein, the method involves preventative or prophylactic treatment. For example, an individual may be at an increased risk of having such a disease or a disorder because of family history of the disease. Typically, individuals at an increased risk of having such a disease or a disorder benefit from prophylactic treatment (e.g., by preventing or delaying the onset or progression of the disease or disorder). In some embodiments, the individual is at an increased risk of having a disease or a disorder caused by aberrant amount of a protein or aberrant activity of a protein.

In some embodiments, a subject treated using the methods and compositions of the invention has received prior treatment for a disease or a disorder (e.g., an immune disease or an immune disorder). Prior treatment may include, e.g., corticosteroids, transplant drugs such as cyclophosphamide and azathioprine, targeted biologics including mAbs blocking TNF, IL-17, IL-12/23 or directed against CD20 (rituximab) or CD52 (alemtuzumab), cytokines including IFN-0, small molecules like dimethyl fumarate and JAK or sphingosine 1 phosphate inhibitors, or intravenous immunoglobulin. In some embodiments, the methods of the present invention are used concurrently with or following other treatments.

In general, suitable routes for administration of ASOs of the present invention will vary depending on the cell type to which delivery of the ASOs is desired. As known to those of skill in the art and described in the literature, different tissues and organs can be affected by disorders (e.g., immune disorders), depending on the disorder and the affected individual. In some embodiments, the ASOs of the present invention are administered to patients parenterally. In some embodiments, the ASOs of the present invention are administered to patients by intracerebroventricular injection, intraperitoneal injection, intramuscular injection, intrathecal injection, subcutaneous injection, oral administration, synovial injection, intravitreal administration, subretinal injection, topical application, implantation, or intravenous injection. In some embodiments, a fetus is treated in utero, e.g., by administering the ASO composition to the fetus directly or indirectly (e.g., via the mother).

In some embodiments, a subject having an immune disorder affecting the brain or CNS, e.g., an autoimmune disorder such as multiple sclerosis, is treated by intrathecal injection, by intracerebroventricular injection, by subcutaneous administration, or by intravenous administration. A subject having an immune disorder affecting the bowel, e.g., an inflammatory disorder such as inflammatory bowel disease, is treated with an oral or subcutaneous administration of the present invention. In some embodiments, inflammatory conditions affecting the liver or kidney inflammation are treated with a subcutaneous or intravenous administration of the present invention. In some embodiments, joint inflammation, e.g., rheumatoid or psoriatic arthritis, is treated with a direct synovial injection or topical administration of the present invention. In some embodiments, inflammation of the skin, e.g., in a subject having psoriasis, is treated with a topical administration of the present invention. In some embodiments, inflammation of the eye, e.g., following corneal transplant or in uveitis, is treated with a topical or intravitreal or subretinal or implant administration of the present invention. In some embodiments, a subject having lupus is treated by subcutaneous administration of the present invention. In some embodiments, the mode of administration is selected to achieve tissue specificity, that is, to upregulate the target protein level (e.g., PD-L1) in target tissue, to minimize the potential for suppression of anti-pathogen immunity. In some embodiments, the appropriate mode of administration is selected by one of skill in the art based on the available literature and his or her knowledge of the given condition.

In some embodiments, the methods and compositions of the present invention are used in combination with an immunosuppressive therapy. An immunosuppressive therapy can comprise any treatment that suppresses the immune system. Immunosuppressive therapy can help to alleviate, minimize, or eliminate transplant rejection in a recipient. For example, immunosuppressive therapy can comprise an immuno-suppressive drug. Immunosuppressive drugs that can be used before, during and/or after transplant, include, e.g., MMF (mycophenolate mofetil (Cellcept)), ATG (anti-thymocyte globulin), anti-CD154 (CD40L), anti-CD40 (2C10, ASKP1240, CCFZ533X2201), alemtuzumab (Campath), anti-CD20 (rituximab), anti-IL-6R antibody (tocilizumab, Actemra), anti-IL-6 antibody (sarilumab, olokizumab), CTLA4-Ig (Abatacept/Orencia), belatacept (LEA29Y), sirolimus (Rapimune), everolimus, tacrolimus (Prograf), daclizumab (Ze-napax), basiliximab (Simulect), infliximab (Remicade), cyclosporin, deoxyspergualin, soluble complement receptor 1, cobra venom factor, compstatin, anti C5 antibody (eculizumab/Soliris), methylprednisolone, FTY720, everolimus, leflunomide, anti-IL-2R-Ab, rapamycin, anti-CXCR3 antibody, anti-ICOS antibody, anti-OX40 antibody, and anti-CD122 antibody. In some embodiments, more than one immunosuppressive agent or drug is used together or sequentially. Immunosuppression can also be achieved using non-drug regimens including, but not limited to, whole body irradiation, thymic irradiation, and full and/or partial splenectomy. These techniques can also be used in combination with one or more immuno-suppressive drugs in conjunction with the methods and compositions of the invention, as appropriate.

Methods of Identifying Additional ASOs that Modulate Splicing

Also, within the scope of the present invention are methods for identifying (determining) additional ASOs that modulate (e.g., inhibit or enhance) splicing of a AIC pre-mRNA, specifically of the alternative-intron. In some embodiments, the target AIC pre-mRNA is encoded by a gene selected from: ARHGAP23, BRD1, DCHS1, EPB41L2, GPX8, HIVEP3, INVS, KIAA0319, NAIP, CD274, PTCH2, PTPRZ1, SON, ZCCHC2. In some embodiments, within the scope of the present invention are methods for identifying (determining) additional ASOs that modulate (e.g., inhibit or enhance) splicing of a CD274 AIC pre-mRNA, specifically of the alternative-intron. ASOs that specifically hybridize to different nucleotides within the target region of the pre-mRNA may be screened to identify (determine) ASOs that modulate (e.g., impair or improve) the rate and/or extent of splicing of the alternative-intron. In some embodiments, the ASO may promote the binding of a splicing repressor(s)/silencer. In some embodiments, the ASO may block or interfere with the binding site(s) of a splicing repressor(s)/silencer. Any method known in the art may be used to identify (determine) an ASO that when hybridized to the target region of the pre-mRNA results in the desired effect (e.g., enhanced protein or functional RNA production). These methods also can be used for identifying ASOs that modulates splicing of the alternative-intron by binding to a targeted region in an intron upstream of a first portion of an exon flanking the 5′ splice site of the alternative-intron, in an intron downstream of a second portion of the exon flanking the 3′ splice site of the alternative-intron, in the first portion of the exon, in the second portion of the exon, or in the alternative-intron. An example of a method that may be used is provided below.

A round of screening, referred to as an ASO “walk” may be performed using ASOs that have been designed to hybridize to a target region of a pre-mRNA. For example, the ASOs used in the ASO walk can be tiled every 5 nucleotides from approximately 100 nucleotides upstream of the 5′ splice site of the alternative-intron (e.g., a portion of sequence of the exon located upstream of the target intron or alternative-intron or a portion of a sequence of a first portion of an exon flanking 5′ splice site of the alternative-intron) to approximately 100 nucleotides downstream of the 5′ splice site of the alternative-intron and/or from approximately 100 nucleotides upstream of the 3′ splice site of the alternative-intron to approximately 100 nucleotides downstream of the 3′ splice site of the alternative-intron (e.g., aportion of sequence of the exon located downstream of the target intron or alternative-intron or a portion of sequence of a second portion of the exon flanking 3′ splice site of the alternative-intron). For example, a first ASO of 15 nucleotides in length may be designed to specifically hybridize to nucleotides +1 to +15 relative to the 5′ splice site of the alternative-intron. A second ASO is designed to specifically hybridize to nucleotides +6 to +20 relative to the 5′ splice site of the alternative-intron. ASOs are designed as such spanning the target region of the pre-mRNA. In some embodiments, the ASOs can be tiled more closely, e.g., every 1, 2, 3, or 4 nucleotides. Further, the ASOs can be tiled from 100 nucleotides downstream of the 5′ splice site, to 100 nucleotides upstream of the 3′ splice site.

One or more ASOs or a control ASO (an ASO with a scrambled sequence, sequence that is not expected to hybridize to the target region) are delivered, for example by transfection, into a disease-relevant cell line that expresses the target pre-mRNA (e.g., the AIC pre-mRNA described elsewhere herein). The splicing-modulating effects (e.g., splicing-inhibiting effects or splicing-inducing effects) of each of the ASOs may be assessed by any method known in the art, for example, by reverse transcriptase (RT)-PCR using primers that span the splice junction. An increase in the RT-PCR product produced using the primers spanning the splice junction in ASO-treated cells as compared to in control ASO-treated cells indicates that splicing of the target intron has been inhibited. A reduction or absence of the RT-PCR product produced using the primers spanning the splice junction in ASO-treated cells as compared to in control ASO-treated cells indicates that splicing of the target intron has been enhanced. RT-PCR may also use primers that flank the splice junction, as described in Example 2. RT-PCR products may be of different sizes, due to the absence or presence of a splicing event or due to a change or an addition of a splice junction. For example, the presence of a smaller RT-PCR product may indicate that an additional splicing event took place or that a splicing event occurred at a different position. The smaller RT-PCR product may indicate that an alternative-intron was spliced out.

In some embodiments, the splicing efficiency, the ratio of spliced to unspliced pre-mRNA, the rate of splicing, or the extent of splicing may be modulated (e.g., impaired or improved) using the ASOs described herein. The amount of protein or functional RNA that is encoded by the target pre-mRNA can also be assessed to determine whether each ASO achieved the desired effect (e.g., enhanced protein production). Any method known in the art for assessing and/or quantifying RNA or protein production, such as qPCR, RT-PCR, Western blotting, flow cytometry, immunofluorescence microscopy, and ELISA, can be used.

A second round of screening, referred to as an ASO “micro-walk” may be performed using ASOs that have been designed to hybridize to a target region of a pre-mRNA. The ASOs used in the ASO micro-walk are tiled every 1 nucleotide to further refine the nucleotide acid sequence of the pre-mRNA that when hybridized with an ASO results in modulated (e.g., inhibited or enhanced) splicing.

Regions defined by ASOs that inhibit or promote splicing of the target intron are explored in greater detail by means of an ASO “micro-walk”, involving ASOs spaced in 1-nucleotide steps, as well as longer ASOs, typically 18-25 nucleotides.

As described for the ASO walk above, the ASO micro-walk is performed by delivering one or more ASOs, or a control ASO (an ASO with a scrambled sequence, sequence that is not expected to hybridize to the target region), for example by transfection, into a disease-relevant cell line that expresses the target pre-mRNA. The splicing-inhibiting effects or splicing-inducing effects of each of the ASOs may be assessed by any method known in the art, for example, by reverse transcriptase (RT)-PCR using primers that span the splice junction. An increase in the RT-PCR product produced using the primers spanning the splice junction in ASO-treated cells as compared to in control ASO-treated cells indicates that splicing of the target intron has been inhibited. A reduction or absence of the RT-PCR product produced using the primers spanning the splice junction in ASO-treated cells as compared to in control ASO-treated cells indicates that splicing of the target intron has been enhanced. RT-PCR may also use primers that flank the splice junction, as described in Example 2. RT-PCR products may be of different sizes, due to the absence or presence of a splicing event or due to a change or a addition of a splice junction. For example, the presence of a smaller RT-PCR product may indicate that an additional splicing event took place or that a splicing event occurred at a different position. The smaller RT-PCR product may indicate that an alternate-intron was spliced. In some embodiments, the splicing efficiency, the ratio of spliced to unspliced pre-mRNA, the rate of splicing, or the extent of splicing may be modulated (e.g., impaired or improved) using the ASOs described herein. The amount of protein or functional RNA that is encoded by the target pre-mRNA can also be assessed to determine whether each ASO achieved the desired effect (e.g., enhanced protein production). Any method known in the art for assessing and/or quantifying RNA or protein production, such as qPCR, RT-PCR, Western blotting, flow cytometry, immunofluorescence microscopy, and ELISA, can be used.

ASOs that when hybridized to a region of a pre-mRNA result in modulated splicing (e.g., inhibited or enhanced splicing) and protein production (e.g., increased or decreased protein production) may be tested in vivo using animal models, for example, transgenic mouse models in which the full-length human gene has been knocked-in or in humanized mouse models of disease. Suitable routes for administration of ASOs may vary depending on the disease and/or the cell types to which delivery of the ASOs is desired. ASOs may be administered, for example, by intracerebroventricular injection, intraperitoneal injection, intramuscular injection, intrathecal injection, subcutaneous injection, oral administration, synovial injection, intravitreal administration, subretinal injection, topical application, implant, or intravenous injection. Following administration, the cells, tissues, and/or organs of the model animals may be assessed to determine the effect of the ASO treatment by for example evaluating splicing (efficiency, rate, or extent) and protein production by methods known in the art and described herein. The animal models may also be any phenotypic or behavioral indication of the disease or disease severity.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The present invention will be more specifically illustrated by the following Examples. However, it should be understood that the present invention is not limited by these examples in any manner.

### Example 1: Identification of Alternative-Introns in CD274 Transcripts by RNAseq Using Next Generation Sequencing

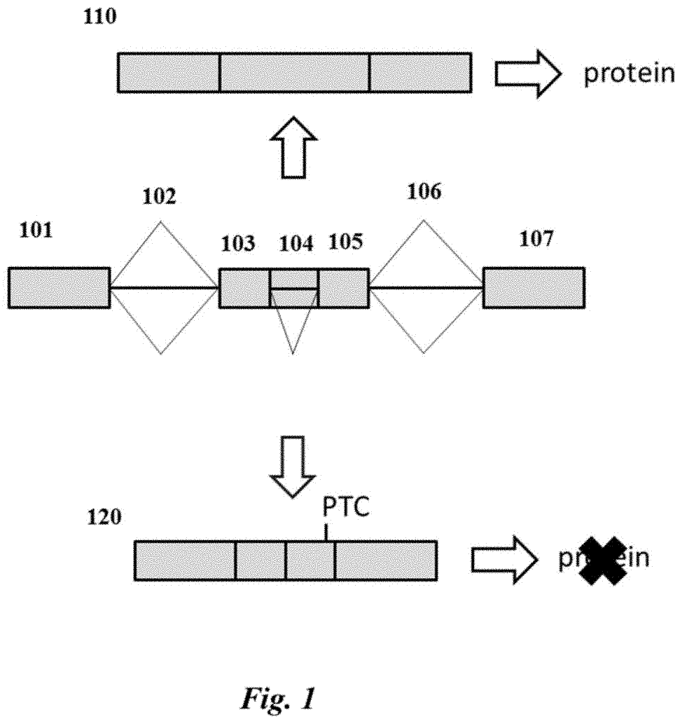

Whole transcriptome shotgun sequencing can be carried out using next generation sequencing to reveal a snapshot of transcripts produced by the CD274 gene to identify alternative-introns. For this purpose, polyA+ RNA from nuclear and cytoplasmic fractions of AST (human astrocyte) cells can be isolated and cDNA libraries constructed using Illumina's TruSeq Stranded mRNA library Prep Kit. The libraries can be pair-end sequenced that can result in 100-nucleotide reads and can be mapped to the human genome. The mapped reads are visualized using the UCSC genome browser (operated by the UCSC Genome Informatics Group (Center for Biomolecular Science & Engineering, University of California, Santa Cruz, 1156 High Street, Santa Cruz, Calif. 95064) and described by, e.g., Rosenbloom, et al., 2015, “The UCSC Genome Browser database: 2015 update,” Nucleic Acids Research 43, Database Issue, doi: 10.1093/nar/gkul 177) and the coverage and number of reads can be inferred by the peak signals. Based on the sequencing reads, Exon 4 in CD274 was identified as having a potential alternative-intron. A schematic of the alternative-intron construct is shown in FIG. 1. The mRNA sequence that encodes a functional protein is labeled as 110. Sequence 110 is composed of 3 exons, 101, 103-105, and 107. The intron 102 and 106 are spliced out and are not part of construction 110. However, an alternate form of the mRNA, labeled as 120, may be generated and results in a premature termination codon (PTC) and therefore does not encode a functional protein. This alternate mRNA is instead composed of 101, 103, 105 and 107, where 104 (which is part of the exon of construct 110) is spliced out as a “alternative-intron”.

### Example 2: In Vitro Observation of Alternative-Intron in Exon 4

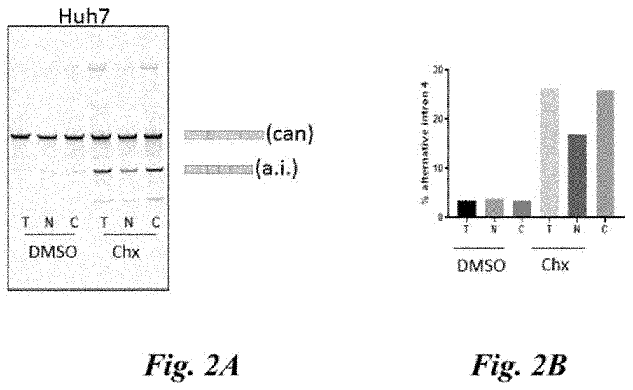

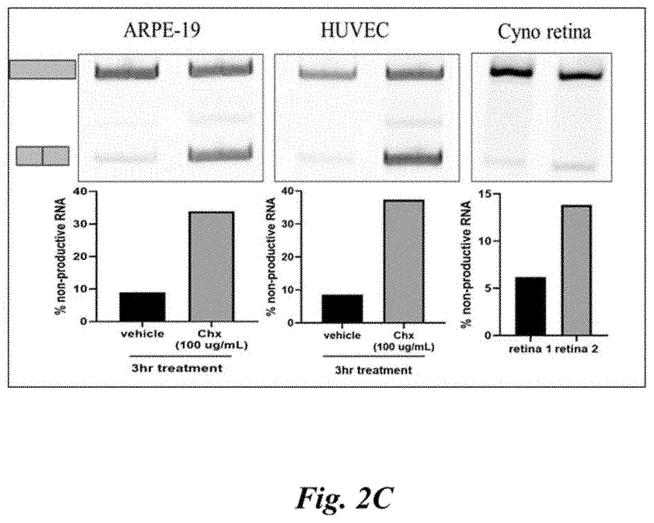

PCR primers are designed with homology to exon 4 and exon 6 and a RT-PCR reaction is performed to generate amplicons pertaining mRNA transcripts in Huh7 cells. An amplification reaction is run using total RNA (labeled T), and RNA from fractionated cells corresponding to the nucleus (labeled N) and the cytoplasm (labeled C) to generate amplicons of mRNA transcripts. The amplicons are run on an polyacrylamide gel and intensities of the different PCR products can be observed. The product that results from the removal of the “alternate intron” (a.i.) is observed to be lower on the gel and of a smaller size than the product corresponding to the the full-length functional mRNA transcript (labeled as can). To visualize the level of the a.i. removal, cells were treated with cycloheximide (CHX) or DMSO control. CHX, a translation inhibitor, inhibits nonsense-mediated mRNA decay which normally degrades the mRNA lacking the alternative intron, as the removal of the a.i. leads to the introduction of a PTC. FIG. 2A shows the gel as well as a schematic of the can and a.i. products. FIG. 2B show the percent abundance of the product resulting from removal of the a.i. relative to total (can+a.i.). FIG. 2C shows the gel, schematics and percent abundance of the product resulting from removal of the a.i. relative to total in two human cell lines (ARPE-19, and HUVEC) and non-human primate retinas (cyno retina).

### Example 3: Design of ASO-Walk Targeting Exon 4 of CD274

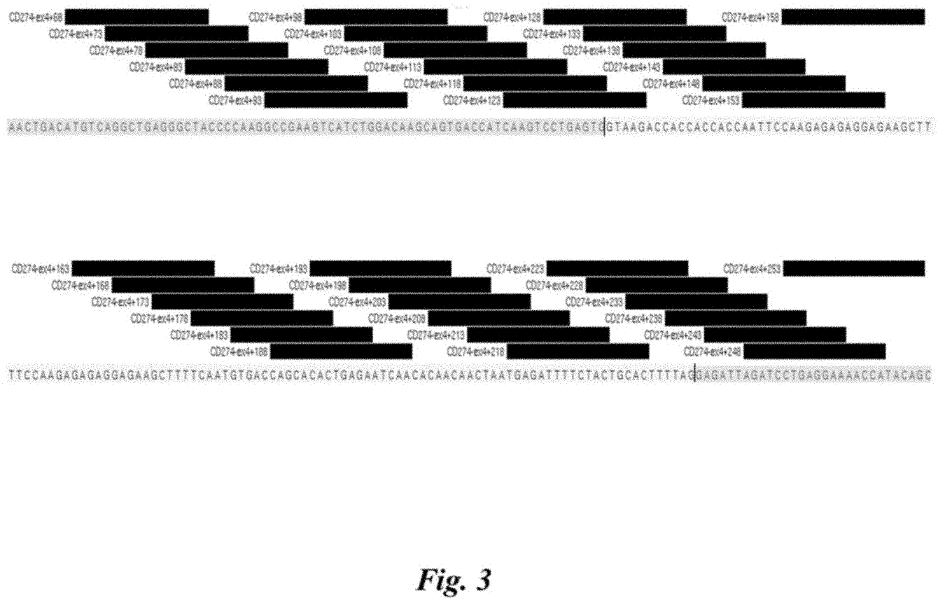

An ASO walk was designed to target exon 4. (SEQ ID NOs:1-67). A region spanning nucleotides +68 to +253 was targeted with 2′-O-MOE RNA, PS backbone, 18-mer ASOs shifted by 5-nucleotide intervals. FIG. 3 shows this ASO walk, with each black box indicating the span of an ASO and the sequence indicated underneath the black block indicating the target sequence in exon 4. Each block spans 18 nucleotides, representing an ASO of 18 nucleotides, and starts 5 nucleotides apart, representing the “walk” of 5-nucleotide intervals.

### Example 4: Screening of ASO-Walk Targeting Exon 4 of CD274

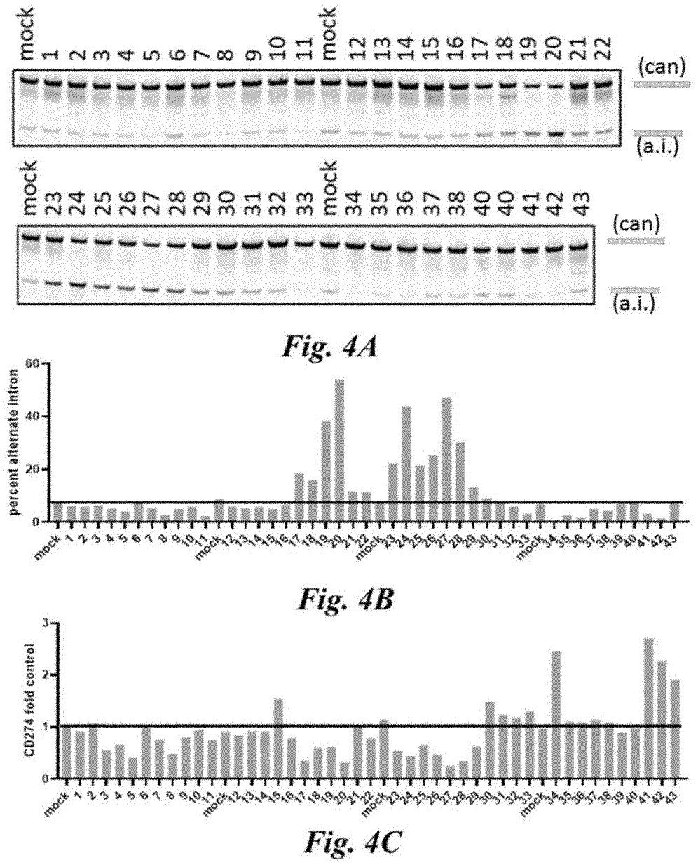

ASOs from example 3 were screened using ARPE-19 cells. ASOs were transfected in ARPE-19 cells for 24 hrs using 80 nM of ASOs depicted in FIG. 3. RT-PCR products were amplified using primers in exon 4 and exon 6 and run on a polyacrylamide gel. FIG. 4A shows a higher band corresponding to the full length can mRNA, and a lower band corresponding to the shorter a.i. mRNA. FIG. 4B shows the quantification of RT-PCR products plotted as a percentage of the alternative intron (a.i./(can+a.i.)*100) ASOs were denoted as possible candidate when the relative abundance of a.i was decreased. Treatment with ASO 34, for example, correlates to a decreased relative abundance compared to a control (mock). FIG. 4C shows Taqman qPCR analysis using RNA from samples in panel A. The Taqman probe is positioned over the exon 3-exon 4 junction. As observed, CD247 mRNA expression has a general inverse correlation with the relative abundance of the a.i. transcript. ASO 34, for example, show an increase in CD247 expression compared to control.

### Example 5: Measuring ASO Activity

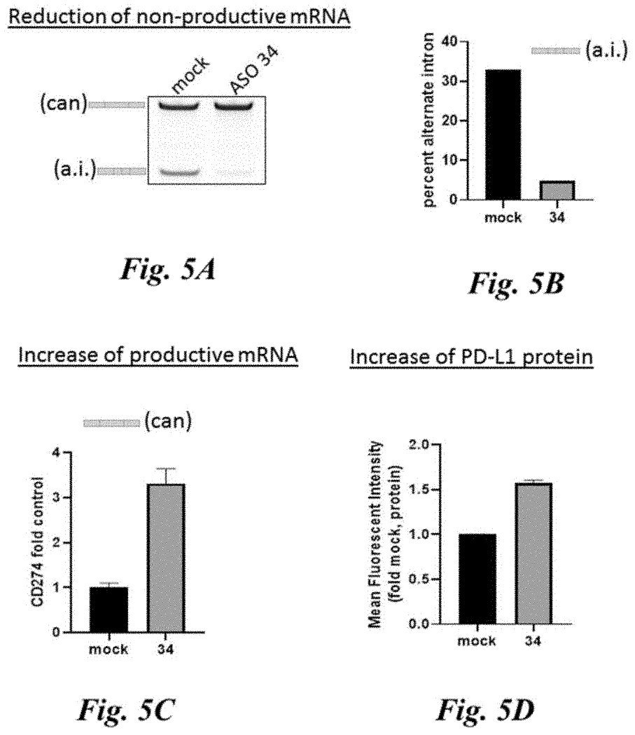

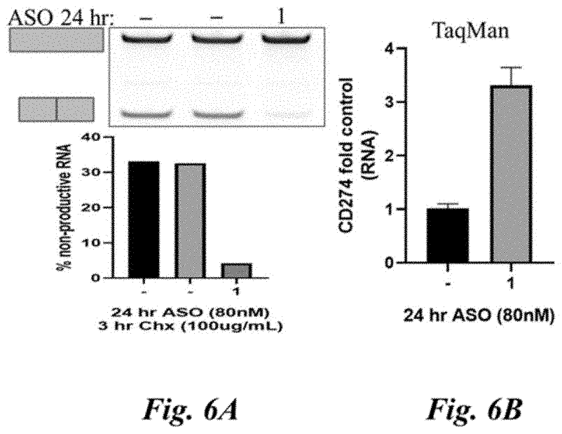

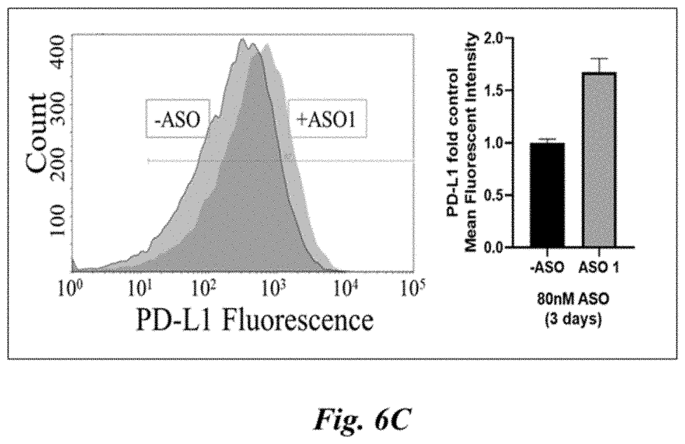

The selected ASO was transfected in Huh7 cells for 21 hr using 80 nM of ASO. Multiple assays were used to determine general activity of the selected ASO. FIG. 5A shows RT-PCR using RNA from Huh7 cells transfected for 21 hours with a selected 80 nM ASO, followed by 3 hours of cycloheximide treatment. Primers were positioned in exons 4 and 6. FIG. 5B shows quantification of RT-PCR products plotted as a percentage of the alternative intron (a.i./(can+a.i.)*100) ASO 34 led to a reduction in the amount of the a.i. product. This is observed in FIG. 5B with the intensity of the band corresponding to a.i. is less in the ASO 34 transfected cells compared to the mock-transfected cells. FIG. 5C shows TaqMan qPCR analysis using RNA from Huh7 cells transfected for 24 hours was used to quantify the activity of 80 nM of ASO compared to a mock. The TaqMan qPCR probe was positioned over the exon 3-4 junction. Results show a higher relative abundance of full length can mRNA in ASO 34 transfected cells as compared to the mock transfected cells. FIG. 5D shows the mean fluorescent intensity of a flow cytometry analysis of Huh7 cells transfected for 5 days with 80 nM ASO 34 plotted as fold change over control (mock) to quantify the expression of PD-L1 protein.

TABLE 2
CD274 Target Sequences

| SEQ ID NO. | REFERENCE |
|---|---|
| 68 | CD274 Exon 4 |
| 69 | CD274 mRNA NCBI Reference Sequence: NM_014143.3 |
| 70 | CD274 Amino Acid Sequence UniProtKB/ |
| | Swiss-Prot: Q9NZQ7-1 |
| 71 | CD274 Genomic Sequence ENST00000381577.3 range = chr9: 5450525-5470547 |
| 72 | CD247 Pre-mRNA sequence hg38_knownGene_ENST00000381573.8 range = chr9: 5450503- |
| | 5470566 |
| 73 | CD247 Pre-mRNA sequence hg38_knownGene_ENST00000381577.3 range = chr9: 5450525- |
| | 5470547 |
| 74 | CD247 Pre-mRNA sequence hg38_knownGene_ENST00000498261.1 range = chr9: 5456114- |
| | 5467866 |
| 75 | CD247 Pre-mRNA sequence hg38_knownGene_ENST00000474218.1 range = chr9: 5457384- |
| | 5463238 |
| 76 | CD247 Pre-mRNA sequence hg38_knownGene_ENST00000492923.1 range = chr9: 5462935- |
| | 5465912 |

### Example 6: Alternative Introns in Various Gene Transcripts

Whole transcriptome shotgun sequencing can be carried out using next generation sequencing to reveal a snapshot of transcripts produced by genes to identify alternative-introns. Cells can be isolated and cDNA libraries constructed using Illumina's TruSeq Stranded mRNA library Prep Kit as also described in Example 1. The libraries can be analyzed and based on the sequencing reads, exons in other genes can be identified as having a potential alternative-intron.

An ASO walk can be designed to target exons identified using 2′-O-MOE RNA, PS backbone, 18-mer ASOs shifted by 5-nucleotide intervals similar to Example 3. An ASO can then be validated via the exemplary screening method of Example 4 and then can be used to prevent or promote de inclusion of an alternate intron event in an mRNA transcript. ASO can be further injected into mouse models to confirm activity and increased expression. Various animal disease models may be used to validate increased expression of the target protein. Table 3 provides some examples of genes that can be targeted in the manner explained above. Genes listed in Table 3 have been known to comprise alternative introns. Chromosomal coordinates of the genes have been provided in addition to the targeted exon. Table 3 also lists some exemplary diseases that may be treated by the increased expression of the genes. Sequences and chromosomal coordinates provided in Table 3 have been generated from the GRCh38/hg38 assembly.

TABLE 3
List of exemplary genes, corresponding target alternative introns, exons, exemplary
corresponding ASOs and diseases to be treated.

| | | | | | Coordinates of | | | | |
|---|---|---|---|---|---|---|---|---|---|
| | | | | | Exon where | | | Exem- | |
| | | | | | Alternative intron | | | plary | |
| | Alternative intron coordinates | | | | is located | | | ASO | Exemplary |
| | | | | SEQ | | SEQ | | SEQ | Diseases to be |
| | | | | ID | | ID | | ID | treated using the |
| Gene | Chrm | Start | End | NO: | Start: End | NO: | Strand | Nos | ASOs |
| ARHGAP23 | chr17 | 38477706 | 38477827 | 77 | 38477579: | 91 | + | 105- | Sclerocystic |
| | | | | | 38477896 | | | 153 | Ovaries; |
| | | | | | | | | | Polycystic Ovary |
| | | | | | | | | | Syndrome |
| BRD1 | chr22 | 49787495 | 49787576 | 78 | 49787390: | 92 | − | 154- | Schizophrenia; |
| | | | | | 49787887 | | | 217 | Bipolar Disorder; |
| | | | | | | | | | Adenoid Cystic |
| | | | | | | | | | Carcinoma |
| DCHS1 | chr11 | 6627026 | 6627254 | 79 | 6626789: | 93 | − | 218- | Van |
| | | | | | 6627667 | | | 334 | Maldergem |
| | | | | | | | | | Wetzburger |
| | | | | | | | | | Verloes |
| | | | | | | | | | syndrome; |
| | | | | | | | | | Mitral Valve |
| | | | | | | | | | Prolapse, |
| | | | | | | | | | Myxomatous |
| | | | | | | | | | 2; Colorectal |
| | | | | | | | | | Cancer; |
| | | | | | | | | | Heterotopia, |
| | | | | | | | | | Periventricular, |
| | | | | | | | | | Autosomal |
| | | | | | | | | | Recessive; |
| | | | | | | | | | Familial |
| | | | | | | | | | mitral valve |
| | | | | | | | | | prolapse |
| EPB41L2 | chr6 | 130869945 | 130870038 | 80 | 130869563: | 94 | − | 335- | Liver Cirrhosis |
| | | | | | 130870126 | | | 406 | |
| GPX8 | chr5 | 55160244 | 55160329 | 81 | 55160169: | 95 | + | 407- | Liver Cirrhosis |
| | | | | | 55160396 | | | 443 | |
| HIVEP3 | chr1 | 41579843 | 41580041 | 82 | 41579737: | 96 | − | 444- | Colorectal Cancer |
| | | | | | 41585318 | | | 1027 | |
| INVS | chr9 | 100292874 | 100292946 | 83 | 100292326: | 97 | + | 1028- | Nephronophthisis |
| | | | | | 100293043 | | | 1112 | 2; Cholestasis; |
| | | | | | | | | | Infantile |
| | | | | | | | | | Cholestasis; Renal |
| | | | | | | | | | dysplasia and |
| | | | | | | | | | retinal aplasia |
| | | | | | | | | | (disorder) |
| KIAA0319 | chr6 | 24596241 | 24596436 | 84 | 24595873: | 98 | − | 1113- | Alexia; |
| | | | | | 24596618 | | | 1212 | Developmental |
| | | | | | | | | | reading disorder; |
| | | | | | | | | | Dyslexia |
| NAIP | chr5 | 70983973 | 70984696 | 85 | 70983775: | 99 | − | 1213- | Muscular |
| | | | | | 70985886 | | | 1501 | Atrophy, Spinal, |
| | | | | | | | | | Type II; Muscular |
| | | | | | | | | | atrophy, spinal, |
| | | | | | | | | | infantile chronic |
| | | | | | | | | | form; Hereditary |
| | | | | | | | | | Motor Neuropathy |
| | | | | | | | | | Proximal Type I; |
| | | | | | | | | | Juvenile Spinal |
| | | | | | | | | | Muscular Atrophy |
| PTCH2 | chr1 | 44827490 | 44827577 | 86 | 44827402: | 100 | − | 1502- | Medulloblastoma; |
| | | | | | 44827714 | | | 1547 | Pigmented Basal |
| | | | | | | | | | Cell Carcinoma; |
| | | | | | | | | | Gastrointestinal |
| | | | | | | | | | Stromal Sarcoma; |
| | | | | | | | | | Oculo-dento- |
| | | | | | | | | | digital syndrome; |
| | | | | | | | | | Medullomyoblastoma; |
| | | | | | | | | | Basal cell |
| | | | | | | | | | carcinoma; |
| | | | | | | | | | Childhood |
| | | | | | | | | | Medulloblastoma; |
| | | | | | | | | | Macrostomia; |
| | | | | | | | | | Desmoplastic |
| | | | | | | | | | Medulloblastoma; |
| | | | | | | | | | Hydrocephalus; |
| | | | | | | | | | Adult |
| | | | | | | | | | Medulloblastoma; |
| | | | | | | | | | Melanotic |
| | | | | | | | | | medulloblastoma; |
| | | | | | | | | | Basal Cell Nevus |
| | | | | | | | | | Syndrome |
| PTPRZ1 | chr7 | 122011310 | 122012607 | 87 | 122010334: | 101 | + | 1548- | Schizophrenia; |
| | | | | | 122013889 | | | 2039 | Pneumoconiosis; |
| | | | | | | | | | Bagassosis |
| SON | chr21 | 33551290 | 33552247 | 88 | 33549476: | 102 | + | 2040- | Malignant |
| | | | | | 33555391 | | | 2733 | neoplasm of |
| | | | | | | | | | salivary gland; |
| | | | | | | | | | ZTTK Syndrome; |
| | | | | | | | | | Adenoid Cystic |
| | | | | | | | | | Carcinoma |
| SON | chr21 | 33551290 | 33552559 | 89 | 33549476: | 103 | + | 2734- | Malignant |
| | | | | | 33555391 | | | 3458 | neoplasm of |
| | | | | | | | | | salivary gland; |
| | | | | | | | | | ZTTK Syndrome; |
| | | | | | | | | | Adenoid Cystic |
| | | | | | | | | | Carcinoma |
| ZCCHC2 | chr18 | 62574927 | 62575312 | 90 | 62574057: | 104 | + | 3459- | Influenza |
| | | | | | 62575550 | | | 3651 | |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1-120. (canceled)

121. A method of modulating expression of a target protein or a target RNA by cells having an alternative-intron-containing pre-mRNA (AIC pre-mRNA), the AIC pre-mRNA comprising an alternative-intron, a first portion of an exon flanking a 5′ splice site of the alternative-intron, a second portion of the exon flanking a 3′ splice site of the alternative-intron, the method comprising contacting the cells with a therapeutic agent or a vector encoding the therapeutic agent,
wherein the therapeutic agent binds to a targeted region of the AIC pre-mRNA encoding the target protein or the target RNA, whereby splicing of the alternative-intron from the AIC pre-mRNA encoding the target protein or the target RNA is modulated, thereby modulating a level of processed mRNA encoding the target protein or the target RNA, and modulating expression of the target protein or the target RNA in the cells.

122. The method of claim 121, wherein the therapeutic agent inhibits exclusion of the alternative-intron from the AIC pre-mRNA and increases a level of a processed mRNA that comprises the alternative-intron, the first portion of the exon and the second portion of the exon, thereby increasing expression of the target protein or the target RNA in the cells.

123. The method of claim 121, wherein the therapeutic agent decreases splicing of the alternative-intron from the AIC pre-mRNA, thereby increasing a level of the processed mRNA encoding the target protein or the target RNA and increasing expression of the target protein or the target RNA in the cells.

124. The method of claim 121, wherein at least a portion of the targeted region of the AIC pre-mRNA is within the alternative-intron, at least a portion of the targeted region of the AIC pre-mRNA is within the first portion of the exon, and/or at least a portion of the targeted region of the AIC pre-mRNA is within the second portion of the exon.

125. The method of claim 121, wherein the target protein expressed from the processed mRNA is a full-length protein and/or a fully functional protein, or wherein the target RNA expressed from the processed mRNA is a fully functional RNA.

126. The method of claim 121, wherein splicing of the alternative-intron from the AIC pre-mRNA encoding the target protein or the target RNA produces a processed mRNA with a premature termination codon (PTC) and/or that undergoes non-sense mediated decay (NMD).

127. The method of claim 121, wherein splicing of the alternative-intron from the AIC pre-mRNA encoding the target protein or the target RNA produces a processed mRNA that encodes a non-functional target protein or a non-functional target RNA

128. The method of claim 121, wherein:
(a) the AIC pre-mRNA is a CD274 pre-mRNA and the target protein is PD-L1 (CD274),
(b) the AIC pre-mRNA is a ARHGAP23 pre-mRNA and the target protein is rho GTPase activating protein 23,
(c) the AIC pre-mRNA is a BRD1 pre-mRNA and the target protein is bromodomain containing 1 protein,
(d) the AIC pre-mRNA is a DCHS1 pre-mRNA and the target protein is protocadherin-16 protein,
(e) the AIC pre-mRNA is a EPB41L2 pre-mRNA and the target protein is band 4.1-like protein 2,
(f) the AIC pre-mRNA is a GPX8 pre-mRNA and the target protein is glutathione peroxidase 8,
(g) the AIC pre-mRNA is a HIVEP3 pre-mRNA and the target protein is human immunodeficiency virus type I enhancer-binding protein 3,
(h) the AIC pre-mRNA is a INVS pre-mRNA and the target protein is inversin,
(i) the AIC pre-mRNA is a KIAA0319 pre-mRNA and the target protein is dyslexia-associated protein KIAA0319 protein,
(j) the AIC pre-mRNA is a NAIP pre-mRNA and the target protein is NLR family apoptosis inhibitory protein,
(k) the AIC pre-mRNA is a PTCH2 pre-mRNA and the target protein is protein patched homolog 2,
(l) the AIC pre-mRNA is a PTPRZ1 pre-mRNA and the target protein is protein tyrosine phosphatase receptor type Z1,
(m) the AIC pre-mRNA is a SON pre-mRNA and the target protein is SON, or
(n) the AIC pre-mRNA is a ZCCHC2 pre-mRNA and the target protein is zinc finger CCHC domain-containing protein 2.

129. The method of claim 121, wherein the targeted region of the AIC pre-mRNA to which the therapeutic agent binds is located within exon 4 of a CD274 pre-mRNA.

130. The method of claim 121, wherein the method comprises contacting the cell with the vector encoding the therapeutic agent, and wherein the vector is a viral vector.

131. The method of claim 121, wherein the method comprises contacting the cell with the therapeutic agent, and wherein the therapeutic agent is an antisense oligomer (ASO).

132. The method of claim 131, wherein the antisense oligomer comprises a backbone modification, a modified sugar moiety, or both.

133. The method of claim 131, wherein the pre-mRNA is an CD274 pre-mRNA, and wherein the antisense oligomer has a sequence that is complementary to a sequence with at least 80% sequence identity to a sequence selected from the group consisting of SEQ ID NO: 68.

134. The method of claim 131, wherein the pre-mRNA is an CD274 pre-mRNA, and wherein the antisense oligomer has a sequence with at least about 80% sequence identity to a sequence selected from the group consisting of SEQ ID NOs: 1-67.

135. The method of claim 128, wherein the pre-mRNA is an CD274 pre-mRNA, and wherein the CD274 pre-mRNA comprises a sequence with at least 80% sequence identity to a sequence selected from the group consisting of SEQ ID NOs: 72-76.

136. The method of claim 128, wherein the pre-mRNA is an CD274 pre-mRNA, and wherein the targeted region is within a sequence with at least 80% sequence identity to a sequence selected from the group consisting of SEQ ID NO: 68.

137. The method of claim 128, wherein the pre-mRNA is an CD274 pre-mRNA, and wherein the targeted region of the pre-mRNA:
(a) comprises at least 10 contiguous nucleobases of SEQ ID NO: 68; or
(b) is within a sequence between a pair of genomic sites selected from the group consisting of GRCh38/hg38: chr9 5462900 and GRCh38/hg38: chr9 5463107.

138. The method of claim 128, wherein the targeted region of the pre-mRNA:
(a) overlaps with a junction of the intron upstream of the first portion of the exon and the first portion of the exon;
(b) overlaps with a junction of the first portion of the exon and the alternative-intron;
(c) overlaps with a junction of the alternative-intron and the second portion of the exon; or
(d) overlaps with a junction of the second portion of the exon and the intron downstream of the second portion of the exon.

139. The method of claim 128, wherein:
(a) the AIC pre-mRNA is a CD274 pre-mRNA, the target protein is PD-L1 (CD274), and the method treats a subject suffering from a disease or condition that comprises an immune disease or an immune disorder;
(b) the AIC pre-mRNA is a ARHGAP23 pre-mRNA, the target protein is rho GTPase activating protein 23, and the method treats a subject suffering from a disease or condition that comprises Sclerocystic Ovaries or Polycystic Ovary Syndrome;
(c) the AIC pre-mRNA is a BRD1 pre-mRNA, the target protein is bromodomain containing 1 protein, and the method treats a subject suffering from a disease or condition that comprises Schizophrenia, Bipolar Disorder, or Adenoid Cystic Carcinoma;
(d) the AIC pre-mRNA is a DCHS1 pre-mRNA, the target protein is protocadherin-16 protein, and the method treats a subject suffering from a disease or condition that comprises Van Maldergem Wetzburger Verloes syndrome, Myxomatous 2 Mitral Valve Prolapse, Colorectal Cancer, Autosomal Recessive Periventricular Heterotopia, or Familial mitral valve prolapse;
(e) the AIC pre-mRNA is a EPB41L2 pre-mRNA, the target protein is band 4.1-like protein 2, and the method treats a subject suffering from a disease or condition that comprises liver cirrhosis;
(f) the AIC pre-mRNA is a GPX8 pre-mRNA, the target protein is glutathione peroxidase 8, and the method treats a subject suffering from a disease or condition that comprises liver cirrhosis;
(g) the AIC pre-mRNA is a HIVEP3 pre-mRNA, the target protein is human immunodeficiency virus type I enhancer-binding protein 3, and the method treats a subject suffering from a disease or condition that comprises Colorectal cancer;
(h) the AIC pre-mRNA is a INVS pre-mRNA, the target protein is inversin, and the method treats a subject suffering from a disease or condition that comprises Nephronophthisis 2, Cholestasis, Infantile Cholestasis, Renal dysplasia, or retinal aplasia;
(i) the AIC pre-mRNA is a KIAA0319 pre-mRNA, the target protein is dyslexia-associated protein KIAA0319 protein, and the method treats a subject suffering from a disease or condition that comprises Alexia, Developmental reading disorder, or Dyslexia;
(j) the AIC pre-mRNA is a NAIP pre-mRNA, the target protein is NLR family apoptosis inhibitory protein, and the method treats a subject suffering from a disease or condition that comprises Spinal Muscular Atrophy Type II, infantile chronic form of spinal Muscular atrophy, Hereditary Motor Neuropathy Proximal Type I, or Juvenile Spinal Muscular Atrophy;
(k) the AIC pre-mRNA is a PTCH2 pre-mRNA, the target protein is protein patched homolog 2 and the method treats a subject suffering from a disease or condition that comprises Medulloblastoma, Pigmented Basal Cell Carcinoma, Gastrointestinal Stromal Sarcoma, Oculo-dento-digital syndrome, Medullomyoblastoma, Basal cell carcinoma, Childhood Medulloblastoma, Macrostomia, Desmoplastic Medulloblastoma, Hydrocephalus, Adult Medulloblastoma, Melanotic medulloblastoma, or Basal Cell Nevus Syndrome,
(l) the AIC pre-mRNA is a PTPRZ1 pre-mRNA, the target protein is protein tyrosine phosphatase receptor type Z1, and the method treats a subject suffering from a disease or condition that comprises Schizophrenia, Pneumoconiosis, or Bagassosis;
(m) the AIC pre-mRNA is a SONpre-mRNA, the target protein is SON, and the method treats a subject suffering from a disease or condition that comprises Malignant neoplasm of salivary gland, ZTTK Syndrome, or Adenoid Cystic Carcinoma; or
(n) the AIC pre-mRNA is a ZCCHC2 pre-mRNA, the target protein is zinc finger CCHC domain-containing protein 2, and the method treats a subject suffering from a disease or condition that comprises influenza.

140. A method of treating a disease or a condition in a subject in need thereof by modulating expression of a target protein or a target RNA in a cell of the subject, comprising: contacting a cell of the subject with a therapeutic agent or a vector encoding the therapeutic agent,
wherein the therapeutic agent modulates splicing of an alternative-intron from an alternative-intron-containing pre-mRNA (AIC pre-mRNA) encoding the target protein or the target RNA, wherein the AIC pre-mRNA comprises the alternative-intron, a first portion of an exon flanking a 5′ splice site of the alternative-intron, a second portion of the exon flanking a 3′ splice site of the alternative-intron,
wherein the therapeutic agent binds to a targeted region of the AIC pre-mRNA encoding the target protein or the target RNA, whereby splicing of the alternative-intron from the AIC pre-mRNA encoding the target protein or the target RNA is modulated, thereby modulating a level of processed mRNA encoding the target protein or the target RNA and modulating the expression of the target protein or the target RNA in the cell of the subject.

141. A composition comprising a therapeutic agent for use in a method of modulating expression of a target protein or a target RNA by cells to treat a disease or a condition in a subject in need thereof, associated with an aberrant protein or an aberrant RNA in the subject, wherein the aberrant protein or aberrant RNA is aberrant in amount or activity in the subject, wherein the therapeutic agent modulates splicing of an alternative-intron-containing pre-mRNA (AIC pre-mRNA) encoding the target protein or the target RNA,
wherein the target protein is:
(a) the aberrant protein;
(b) a protein which functionally activates or deactivates cellular signaling mechanisms to alter cellular activity associated with the disease or condition;
(c) a protein which functionally augments or replaces the aberrant protein in the subject; or
(d) a protein which functionally decreases or inhibits the aberrant protein in the subject;
and wherein the target RNA is:
(a) the aberrant RNA;
(b) an RNA which functionally activates or deactivates cellular signaling mechanisms to alter cellular activity associated with the disease or condition;
(c) an RNA which functionally augments or replaces the aberrant RNA in the subject; or
(d) an RNA which functionally decreases or inhibits the aberrant RNA in the subject;
wherein the AIC pre-mRNA comprising an alternative-intron, a first portion of an exon flanking a 5′ splice site of the alternative-intron, a second portion of the exon flanking a 3′ splice site of the alternative-intron, and whereby splicing of the alternative-intron from the AIC pre-mRNA encoding the target protein or the target RNA is modulated, thereby modulating production or activity of the target protein or the target RNA in the subject.
